(19) United States
(12) Patent Application Publication
(10) Pub. No.: US 2025/0108167 A1
(43) Pub. Date: Apr. 3, 2025

(54) MULTI-COMPONENT DELIVERY SYSTEMS WITH INTERNAL NEEDLE AND MICRO SEAL ASSEMBLY

(71) Applicant: CLEARSTREAM TECHNOLOGIES LIMITED, Enniscorthy (IE)

(72) Inventors: Matthew Cullen, Wicklow (IE); Victor Bell, Dublin (IE)

(73) Assignee: CLEARSTREAM TECHNOLOGIES LIMITED, Enniscorthy (IE)

(21) Appl. No.: 18/898,879

(22) Filed: Sep. 27, 2024

Related U.S. Application Data
(60) Provisional application No. 63/586,160, filed on Sep. 28, 2023.
(60) Provisional application No. 63/586,157, filed on Sep. 28, 2023.

(51) Int. Cl. A61M 5/19; A61M 5/28; A61M 5/32

(52) U.S. Cl. CPC A61M 5/19; A61M 5/285; A61M 5/3294

(57) ABSTRACT

A system for mixing and delivering content is disclosed. The system includes an applicator, a needle assembly, and a micro seal. The applicator includes two component chambers separate from one another, each component chamber including at least one output port on a distal end thereof and an applicator hub disposed on a distal end of the applicator adjacent to the at least one output port of each component chamber. The needle assembly includes an elongate hollow stylet having a plurality of lumens therein and a needle hub having an interior cannula fluidly coupled to the elongate hollow stylet. The micro seal is disposed within the applicator hub. The micro seal is configured to receive the interior cannula of the needle hub to establish a fluid coupling between each of the two component chambers with a respective one of the lumens within the elongate hollow stylet.

### CROSS-REFERENCE TO RELATED APPLICATIONS

The present application claims the benefit of U.S. Provisional Application No. 63/586,157, entitled MIXING SYRINGE ASSEMBLIES AND METHODS OF MIXING CONSTITUENT MATERIALS, and filed Sep. 28, 2023, the entirety of which is hereby incorporated by reference. The present application further claims the benefit of U.S. Provisional Application No. 63/586,160, entitled MULTI-COMPONENT DELIVERY SYSTEMS WITH INTERNAL NEEDLE AND MICRO SEAL ASSEMBLY, and filed Sep. 28, 2023, the entirety of which is hereby incorporated by reference.

### BACKGROUND

### Field

The present disclosure generally relates to multi-component delivery systems, and more specifically, to components for connecting components of multi-component delivery systems.

### Technical Background

A number of procedures utilize dual barrel syringes for component delivery and/or removal. However, for some multi-component materials, it may be necessary to ensure that the syringes do not leak and/or mix prematurely.

### SUMMARY

According to at least one aspect of the present disclosure, a system for mixing and delivering content is disclosed. The system includes an applicator, a needle assembly, and a micro seal. The applicator includes two component chambers separate from one another, each component chamber including at least one output port on a distal end thereof and an applicator hub disposed on a distal end of the applicator adjacent to the at least one output port of each component chamber. The needle assembly includes an elongate hollow stylet having a plurality of lumens therein and a needle hub having an interior cannula fluidly coupled to the elongate hollow stylet. The micro seal is disposed within the applicator hub. The micro seal is configured to receive the interior cannula of the needle hub to establish a fluid coupling between each of the two component chambers with a respective one of the lumens within the elongate hollow stylet.

According to at least one aspect of the present disclosure, a system for mixing and delivering content is disclosed. The system includes a first applicator, a second applicator, and a micro seal. The first applicator includes a first component chamber including a first output port defined on a distal end thereof, a second component chamber separate from the first component chamber, the second component chamber including a second output port defined on a distal end thereof, and an applicator hub disposed on a distal end of the first applicator adjacent to the first output port and the second output port. The second applicator includes a third component chamber including a third output port defined on a distal end thereof, a fourth component chamber separate from the third component chamber, the fourth component chamber including a fourth output port defined on a distal end thereof, and a mixing tube fluidly coupled to the third output port of the third component chamber. The micro seal is disposed within the applicator hub of the first applicator, wherein the micro seal is configured to receive at least a portion of the mixing tube of the second applicator to establish a first fluid pathway between the first component chamber of the first applicator and the third component chamber of the second applicator separate from a second fluid pathway between the second component chamber of the first applicator and the fourth component chamber of the second applicator.

According to at least one aspect of the present disclosure, a system for mixing and delivering content is disclosed. The system includes a first applicator, a second applicator, a needle assembly, and a micro seal. The first applicator includes a first component chamber including a first output port defined on a distal end thereof, a second component chamber separate from the first component chamber, the second component chamber including a second output port defined on a distal end thereof, and an applicator hub disposed on a distal end of the first applicator adjacent to the first output port and the second output port. The second applicator is removably coupled to the first applicator, wherein the second applicator includes a third component chamber including a third output port defined on a distal end thereof, a fourth component chamber separate from the third component chamber, the fourth component chamber including a fourth output port defined on a distal end thereof, and a mixing tube fluidly coupled to the third output port of the third component chamber. The needle assembly includes an elongate hollow stylet having a plurality of lumens therein and a needle hub having an interior cannula fluidly coupled to the elongate hollow stylet, wherein the needle assembly is removably coupled to the distal end of the first applicator in lieu of the second applicator. The micro seal is disposed within the applicator hub of the first applicator, wherein the micro seal is configured to receive at least a portion of the mixing tube of the second applicator when the second applicator is coupled to the first applicator to establish a first fluid pathway between the first component chamber of the first applicator and the third component chamber of the second applicator separate from a second fluid pathway between the second component chamber of the first applicator and the fourth component chamber of the second applicator, and wherein the micro seal is configured to receive the interior cannula of the needle hub when the needle assembly is coupled to the first applicator to establish a fluid coupling between the first component chamber and the second component chamber with a respective one of the lumens within the elongate hollow stylet.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 depicts a cutaway view of an illustrative delivery system including an applicator and an injection needle assembly according to one or more aspects shown and described herein;

FIG. 2 depicts an illustrative needle hub according to one or more aspects shown and described herein;

FIG. 3 depicts an illustrative micro seal disposed within an applicator hub according to one or more aspects shown and described herein;

FIG. 4 depicts an illustrative micro seal according to one or more aspects shown and described herein;

FIG. 5A depicts an exploded cutaway view of a needle hub and an applicator hub according to one or more aspects shown and described herein;

FIG. 5B depicts a cutaway view of the needle hub and the applicator hub of FIG. 5A when joined together according to one or more aspects shown and described herein;

FIG. 6A depicts a perspective cutaway view of an applicator hub with a micro seal according to one or more aspects shown and described herein;

FIG. 6B depicts a cutaway side view of the applicator hub with the micro seal of FIG. 6A according to one or more aspects shown and described herein;

FIG. 7 depicts a cutaway side view of an illustrative delivery system according to one or more aspects shown and described herein;

FIG. 8 depicts a cutaway side view of an illustrative mixing system including an applicator having a mixing tube selectively coupled to a micro seal to allow fluid flow according to one or more aspects shown and described herein;

FIG. 9 depicts an illustrative cutaway view applicator having a mixing tubing positioned therein for connection to a micro seal to allow fluid flow according to one or more aspects shown and described herein;

FIG. 10 depicts an illustrative cutaway view of an applicator having a central wall defined through a body thereof and a plunger according to one or more aspects shown and described herein;

FIG. 11A depicts an illustrative side view of the applicator of FIG. 10 according to one or more aspects shown and described herein;

FIG. 11B depicts an illustrative cutaway view of the applicator along the central wall of FIG. 10 according to one or more aspects shown and described herein;

FIG. 12A depicts an illustrative side view of the body of the applicator of FIG. 10 according to one or more aspects shown and described herein;

FIG. 12B depicts an illustrative cutaway view of the body of the applicator of FIG. 10 according to one or more aspects shown and described herein;

FIG. 12C depicts a perspective cutaway view of the body of the applicator of FIG. 10 according to one or more aspects shown and described herein;

FIG. 12D depicts an illustrative cutaway view of the applicator having the central wall of FIG. 10 according to one or more aspects shown and described herein;

FIG. 12E depicts an end view of the applicator of FIG. 10 according to one or more aspects shown and described herein;

FIG. 12F depicts an end view of the applicator of FIG. 10 according to one or more aspects shown and described herein;

FIG. 13A depicts a perspective view of a plunger for use with a body of an applicator having a central wall defined therethrough according to one or more aspects shown and described herein;

FIG. 13B depicts a top view of the plunger of FIG. 13A according to one or more aspects shown and described herein;

FIG. 13C depicts a side view of the plunger of FIG. 13A according to one or more aspects shown and described herein;

FIG. 14A depicts a perspective view of a plunger for use with a body of an applicator having a central wall defined therethrough according to one or more aspects shown and described herein;

FIG. 14B depicts a perspective view of the plunger of FIG. 14A according to one or more aspects shown and described herein;

FIG. 15 depicts the plunger of FIG. 14A movably positioned within a body of an applicator according to one or more aspects shown and described herein;

FIG. 16 depicts an applicator including ball valves positioned therein to prevent backflow of contents according to one or more aspects shown and described herein;

FIG. 17A depicts a perspective view of a proximal component of a housing of the ball valve of FIG. 16 according to one or more aspects shown and described herein;

FIG. 17B depicts a bottom view of the proximal component of FIG. 17A according to one or more aspects shown and described herein;

FIG. 17C depicts a cutaway view of an intermediate component of a housing of the ball valve of FIG. 16 according to one or more aspects shown and described herein;

FIG. 17D depicts a perspective view of a distal component of a housing of the ball valve of FIG. 16 according to one or more aspects shown and described herein;

FIG. 18 depicts a cutaway view of the ball valves of FIG. 16 in an open configuration according to one or more aspects shown and described herein;

FIG. 19A depicts an illustrative applicator hub according to one or more aspects shown and described herein;

FIG. 19B depicts a perspective view of the applicator hub of FIG. 19A according to one or more aspects shown and described herein;

FIG. 19C depicts a cutaway view of the applicator hub of FIG. 19A according to one or more aspects shown and described herein;

FIG. 20A depicts a mixing assembly including a first applicator and a second applicator coupled to one another via a connector according to one or more aspects shown and described herein;

FIG. 20B depicts an end view of the connector of FIG. 20A according to one or more aspects shown and described herein;

FIG. 20C depicts a side view of the connector of FIG. 20A according to one or more aspects shown and described herein;

FIG. 21A depicts an illustrative dual-barrel applicator having a mixing connector coupled to a distal end thereof according to one or more aspects shown and described herein;

FIG. 21B depicts a cutaway view of the dual-barrel applicator of FIG. 21A having the mixing connector coupled to the distal end thereof according to one or more aspects shown and described herein;

FIG. 21C depicts a detailed cutaway view of the mixing connector of FIG. 21A coupled to the distal end of the applicator according to one or more aspects shown and described herein;

FIG. 21D depicts a detailed cutaway view of the mixing connector of FIG. 21A coupled to the distal end of the applicator according to one or more aspects shown and described herein;

FIG. 22A depicts a partial cutaway view of a mixing connector coupled to a distal end of an applicator according to one or more aspects shown and described herein;

FIG. 22B depicts a partial illustrative view of the mixing connector of FIG. 22A coupled to the distal end of the applicator according to one or more aspects shown and described herein;

FIG. 22C depicts an illustrative perspective view of the mixing connector of FIG. 22A according to one or more aspects shown and described herein;

FIG. 22D depicts an illustrative perspective view of the mixing connector of FIG. 22A according to one or more aspects shown and described herein;

FIG. 23A depicts a mixing assembly including a first applicator and a second applicator coupled to one another through a luer lock connection according to one or more aspects shown and described herein;

FIG. 23B depicts a detailed cutaway view of the connection between the first applicator and the second applicator of FIG. 23A according to one or more aspects shown and described herein;

FIG. 23C depicts a detailed illustrative view of the connection between the first applicator and the second applicator of FIG. 23A according to one or more aspects shown and described herein;

FIG. 24A depicts an illustrative perspective view of an applicator hub according to one or more aspects shown and described herein;

FIG. 24B depicts a cutaway perspective view of the applicator hub of FIG. 24A according to one or more aspects shown and described herein;

FIG. 24C depicts a cutaway side view of the applicator hub of FIG. 24A according to one or more aspects shown and described herein;

FIG. 25A depicts an illustrative side view of the applicator hub of FIG. 24A coupled to a distal end of an applicator according to one or more aspects shown and described herein;

FIG. 25B depicts a cutaway side view of the applicator hub coupled to the distal end of the applicator of FIG. 25A according to one or more aspects shown and described herein;

FIG. 26A depicts an illustrative perspective view of an applicator hub according to one or more aspects shown and described herein; and

FIG. 26B depicts a cutaway perspective view of the applicator hub of FIG. 26A according to one or more aspects shown and described herein.

### DETAILED DESCRIPTION

The present disclosure, in one form, is related to mixing and delivery systems for multi-component materials, such as multi-component sealants, multi-component spacers, and/or the like. The delivery systems described herein include components that deliver the multi-component materials along an access path. The delivery systems described herein include a dual chamber applicator and an injection needle assembly. The injection needle assembly includes a needle hub and an applicator hub that nest together to connect various components. A micro seal disposed within the applicator hub provides a tight seal around various components to ensure separation of multi-component materials as they travel from the dual chamber applicator to a distal end of a needle. The micro seal, in being disposed within the applicator hub, avoids issues associated with seal devices that are externally located (e.g., located around an exposed needle when components are disconnected from one another) such as, for example, falling off, getting caught on equipment such as surgical gloves, and/or the like, and thus represents an improvement over such externally located seal devices. As will be described in greater detail herein, the micro seal may be particularly shaped, sized, and constructed to be placed within an applicator hub and to receive a channel or needle extending from a needle hub to seal the channel or needle and ensure separation of components.

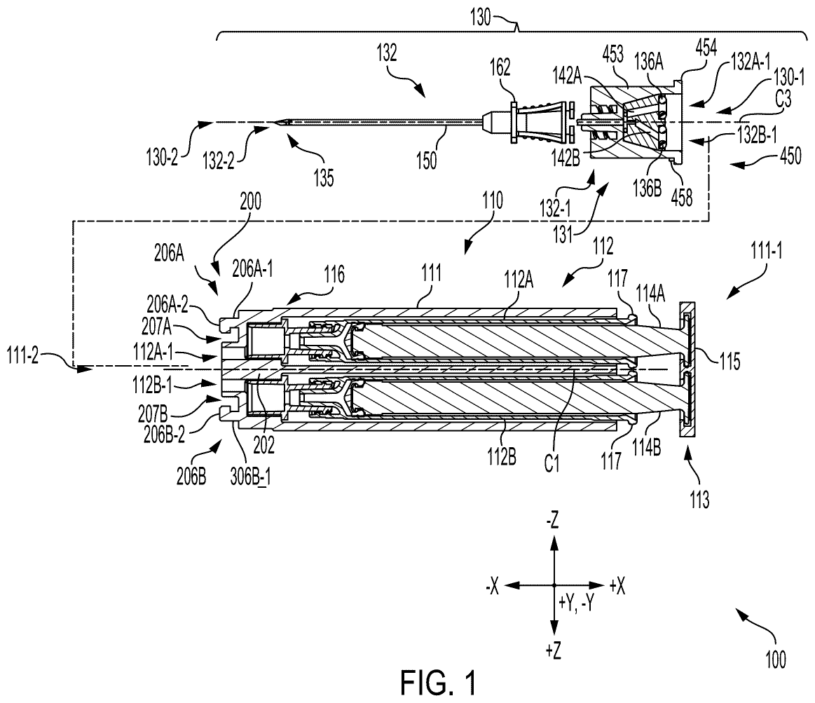

Turning now to the drawings, FIG. 1 depicts an illustrative delivery system 100 according to various embodiments. The delivery system 100 generally includes an applicator 110 and/or an injection needle assembly 130. The various components for the delivery system 100 are keyed via a connection system (e.g., a quarter turn connection system) such that the applicator 110 can be coupled to the injection needle assembly 130 for mixing and delivery of components, as indicated by the dashed lines in FIG. 1 and described herein. When coupled to the injection needle assembly 130, the combination of the injection needle assembly 130 and the applicator 110 may be referred to herein as a delivery apparatus. When the applicator 110 is coupled to the injection needle assembly 130, the delivery apparatus has a configuration to facilitate delivery of a flowable multi-component material to an injection site.

In embodiments, the delivery apparatus may be used in conjunction with an introducer cannula (not shown). The introducer cannula may facilitate withdrawal of the injection needle assembly 130 of the delivery apparatus from a subject, while the introducer cannula may remain in place to maintain the access path in the tissue to the site, such as for example, to receive and guide a second medical instrument, such as a biopsy device, to the site where the biopsy is to be performed.

The applicator 110 generally includes a body 111 having a proximal end 111-1 and a distal end 111-2 spaced a distance apart from the proximal end 111-1. The body 111 also defines a pair of syringes 112. The applicator 110 is configured to separately carry each of a first component of the multi-component material and a second component of the multi-component material. In some embodiments, the material may be a sealant and the first component may include, for example, at least two N-hydroxysuccinimide (NHS) ester groups, and the second component may include, for example, at least two amine groups. For example, the first component may be a solution containing polyethylene glycol (PEG) succinimidyl succinate and the second component may be a solution containing albumin and/or polyethylenimine (PEI). In the present embodiment, the first component and the second component are combined and mixed within a mixing chamber of the injection needle assembly 130, as will be described in greater detail herein.

The pair of syringes 112 includes an actuator 113, a first component chamber 112A, and a second component chamber 112B. The first component chamber 112A may be, for example, a cylindrical tube that is configured to carry the first component of the multi- component sealant. The first component chamber 112A has at least one output port, such as a first output port 112A-1 (e.g., a first component port). The second component chamber 112B also may be, for example, a cylindrical tube that is configured to carry the second component of the multi-component material. The second component chamber 112B has at least one output port, such as a second output port 112B-1 (e.g., a second component port). In some aspects, the first component chamber 112A and the second component chamber 112B are arranged in a substantially longitudinally parallel arrangement. However, the present disclosure is not limited to such. That is, in other embodiments, the first component chamber 112A and the second component chamber 112B can be arranged in other manners, such as, for example, a nested configuration, a stacked configuration, or the like.

In some aspects, the actuator 113 includes a first piston 114A, a second piston 114B, and a handle 115. In some embodiments, the handle 115 is in the form of a link member that perpendicularly extends between, and is connected to, each of the first piston 114A and the second piston 114B to facilitate simultaneous movement of the first piston 114A and the second piston 114B with the depression or retraction of the handle 115. The first piston 114A is in the form of a plunger that is positioned in the first component chamber 112A proximal to the first component, and the second piston 114B is in the form of a plunger that is positioned in the second component chamber 112B proximal to the second component. In other embodiments, the handle 115 may be an overmolded portion of the proximal ends of the first piston 114A and the second piston 114B so as to form a unitary piece. In still other embodiments, the handle 115 may be formed by a integral joining of the proximal ends of the first piston 114A and the second piston 114B so as to form a unitary piece

The first output port 112A-1 of the first component chamber 112A and the second output port 112B-1 of the second component chamber 112B may be arranged within the distal end 111-2 of the body 111 of the applicator 110. The first output port 112A-1 and the second output port 112B-1 are generally fluid outputs that are aligned with other ports of other components as described herein such that the first and second components can be dispensed from and/or received within the respective component chambers 112A, 112B. In some aspects, the first output port 112A-1 may be concentrically aligned with the first component chamber 112A and the second output port 112B-1 may be concentrically aligned with the second component chamber 112B. However, in other aspects, such as the aspect depicted in FIG. 1, the first output port 112A-1 may be located radially inward of a central area of the first component chamber 112A and the second output port 112B-1 may be located radially inward of a central area of the second component chamber 112B such that the first output port 112A-1 and the second output port 112B-1 are as close as possible to a center axis C1 of the body 111 of the applicator 110 to facilitate alignment with the other components of the delivery system 100 described herein.

Still referring to FIG. 1, the body 111 of the applicator 110 further includes a connector 200 integrated with the distal end 111-2 of the applicator 110. More specifically, as depicted in FIG. 1, the various components of the connector 200 are integrated with the body 111 such that the connector 200 and the body 111 are a single monolithic piece. However, it should be understood that this is merely illustrative and the various components of the connector 200 may be separate pieces that are permanently or semi-permanently joined with the body 111 of the applicator 110 (e.g., permanently or semi-permanently joined with a distal coupling piece 116 of the applicator 110).

The connector 200 is generally located at the distal end 111-2 of the body 111 of the applicator 110 such that various components of the connector 200 are positioned adjacent to first output port 112A-1 and the second output port 112B-1. As will be described herein, the connector 200 is generally shaped and sized to releasably interlock with a corresponding connector 450 of the injection needle assembly 130. As will be described in greater detail herein, when the injection needle assembly 130 is coupled to the applicator 110 via the connectors 200, 450 thereof, the various ports thereof are aligned and sealed with the first output port 112A-1 and the second output port 112B-1 of the applicator 110.

In some embodiments, the connector 200 of the applicator includes a circular protrusion 202 extending distally (e.g., in the −x direction of the coordinate axes of FIG. 1) from the distal end 111-2 of the applicator 110, a semi-circular channel (not depicted) disposed within the distal end 111-2 of the applicator 110 along a periphery of the circular protrusion 202, and a pair of bayonet coupling members (e.g., a first coupling member 206A and a second coupling member 206B) disposed radially outward of the circular protrusion 202 and the semi-circular channel. As shown in FIG. 1, the first output port 112A-1 and the second output port 112B-1 are disposed within the circular protrusion 202. That is, the openings into the first component chamber 112A and the second component chamber 112B are located on the circular protrusion 202.

The circular protrusion 202 is generally shaped and sized to correspond to a recess formed in the injection needle assembly 130, as described in greater detail herein. The circular protrusion 202 may generally be disposed in or around a central area of the distal end 111-2 of the body 111. In some embodiments, the circular protrusion 202 may be concentric with the body such that the center axis C1 of the body 111 extends through a center of the circular protrusion 202. The distance that the circular protrusion 202 extends away from the distal end 111-2 of the body is generally a distance that corresponds to a depth of the recess formed in the injection needle assembly 130 such that the circular protrusion 202 can be completely inserted therein, but is otherwise not limited by the present disclosure.

Referring again to FIG. 1, the first coupling member 206A and the second coupling member 206B each extend from the distal end 111-2 of the body 111 of the applicator 110 and are generally shaped and sized to retain the injection needle assembly 130 when coupled to the applicator 110. Each of the first coupling member 206A and the second coupling member 206B may be a bayonet style coupling member, an L-beam coupling member, or the like. For example, as particularly depicted in FIG. 1, the first coupling member 206A extends distally at a particular distance from the distal end 111-2 of the body 111, turns about 90 degrees, and extends inward toward the center axis C1 of the body 111, resulting in a first extension piece 206A-1 that extends in a direction generally coplanar with the center axis C1 of the body 111 (e.g., along the x-axis of the coordinate axes of FIG. 1) and a second extension piece 206A-2 that extends in a direction that is generally perpendicular to the center axis C1 of the body (e.g., along the z-axis of the coordinate axes of FIG. 1), thereby defining a pocket 207A between the distal end 111-2 of the body 111 and the second extension piece 206A-2. Similarly, the second coupling member 206B extends distally at a particular distance from the distal end 111-2 of the body 111, turns about 90 degrees, and extends inward toward the center axis C1 of the body 111, resulting in a first extension piece 206B-1 that extends in a direction generally coplanar with the center axis C1 of the body 111 (e.g., along the x-axis of the coordinate axes of FIG. 1) and a second extension piece 206B-2 that extends in a direction that is generally perpendicular to the center axis C1 of the body 111 (e.g., along the z-axis of the coordinate axes of FIG. 1), thereby defining a pocket 207B between the distal end 111-2 of the body 111 and the second extension piece 206B-2.

As depicted in FIG. 1, the first coupling member 206A and the second coupling member 206B are located opposite one another, radially outward of the circular protrusion 202. However, this is merely illustrative, and other locations and spacing are contemplated and included within the scope of the present disclosure. Further, while the present aspect includes a pair of coupling members (e.g., the first coupling member 206A and the second coupling member 206B), this is also merely illustrative. That is, other amounts of coupling members are also contemplated and included within the scope of the present disclosure.

Still referring to FIG. 1, the injection needle assembly 130 has a proximal end 130-1 that extends proximally (in the +x direction of the coordinate axes of FIG. 1) and a distal end 130-2 that extends distally (e.g., in the −x direction of the coordinate axes of FIG. 1). The injection needle assembly 130 generally includes an applicator hub 131 and an elongate hollow stylet 132 that extends distally (e.g., in the-x direction of the coordinate axes of FIG. 1) from the applicator hub 131. The elongate hollow stylet 132 has a proximal end 132-1 and a distal end 132-2. The applicator hub 131 is fixedly attached, e.g., through overmolding, adhesive and/or pressed fit, to the proximal end 132-1 of the elongate hollow stylet 132. The applicator hub 131 includes a plurality of input ports (e.g., a first input port 132A-1 and a second input port 132B-1) of the injection needle assembly 130. The applicator hub 131 is configured for removable connection to the applicator 110 via the corresponding connector 450 such that, when connected, the first input port 132A-1 of the injection needle assembly 130 is aligned and sealed with the first output port 112A-1 of the applicator 110 and the second input port 132B-1 of the injection needle assembly 130 is aligned and sealed with the second output port 112B-1 of the applicator 110, as described in greater detail herein.

The elongate hollow stylet 132 of injection needle assembly 130 is configured to facilitate fluid communication with the plurality of output ports 112A-1, 112B-1 of the dual chamber applicator 110 so as to receive the two precursor materials of the hydrogel spacer from dual chamber applicator 110 and direct the two components to the distal end 132-2 thereof for mixing and delivery. In addition, the elongate hollow stylet 132 of the injection needle assembly is configured to facilitate fluid communication with the output port 122A-1 of the auxiliary applicator 120 so as to receive the contents of the auxiliary applicator 120 and direct the contents to the distal end 132-2 thereof for delivery. The distal end 132-2 may include a closed distal end or an open distal portion. In addition, the distal end 132-2 may include a plurality of side ports (e.g., two, three, or more) proximal to the closed distal end or a distal port.

While not depicted herein, the elongate cannula 150 of the elongate hollow stylet 132 includes a plurality of lumens. More specifically, the elongate cannula 150 defines an outer side wall that surrounds an outer lumen of the elongate hollow stylet 132. In addition, located within the outer lumen of the elongate cannula 150 is a first inner side wall that surrounds one or more interior lumens, each having a cross sectional size that gets smaller as one traverses from the outer side wall inward. In some embodiments, the one or more interior lumens may be concentric with the outer lumen. Distal openings of each of the lumens are all open to a distal chamber of the elongate hollow stylet 132. A technical effect is thus realized in which each of the lumens maintains a separation of materials until the materials reach the distal chamber. This separation of materials is further maintained via the micro seal, as described in further detail herein.

In some aspects, various distal ports may be positioned such that the flowable multi-component hydrogel may be mixed at the distal end 132-2 of the elongate hollow stylet 132 and delivered out the distal end 132-2 of the elongate hollow stylet 132, so as to ensure spacer placement in a targeted area.

In an embodiment, the features of the connectors 200, 450 may advantageously result in a structure that allows a user to quickly connect and subsequently disconnect components (e.g., the applicator 110 with the injection needle assembly 130) in such a manner that a user can easily confirm that components are correctly sealed and aligned with one another to ensure the correct materials held within are mixed together and then subsequently delivered to a site on a subject. This improvement is shown, for example, in the quarter turn operation of the various components of the delivery system 100 (FIG. 1), but is not limited to such. That is, other quick connect/disconnect components are also contemplated and included in the scope of the present disclosure.

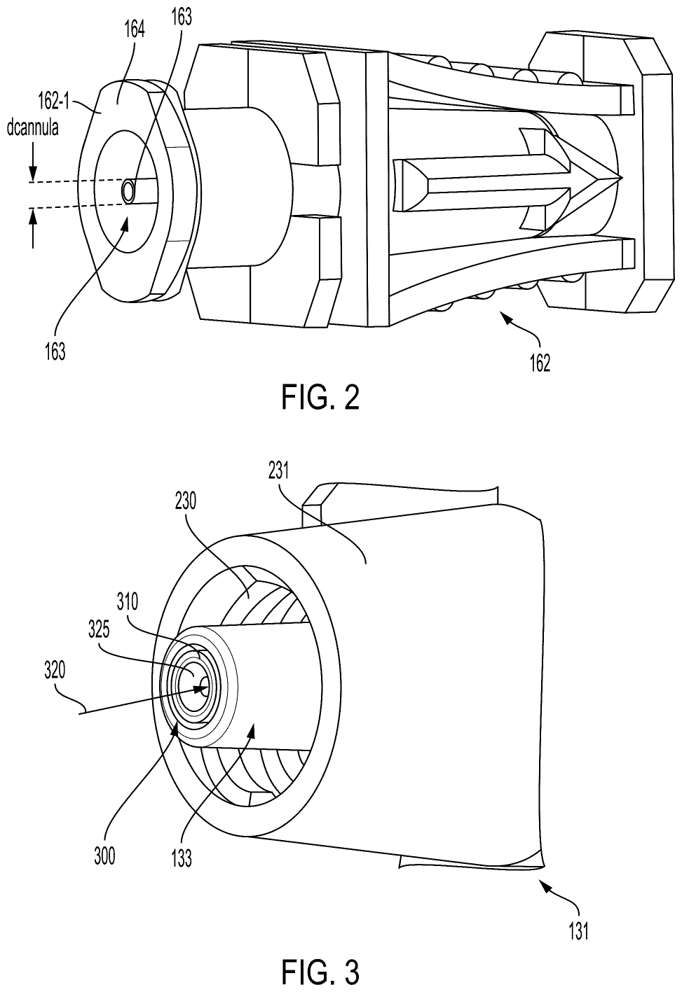

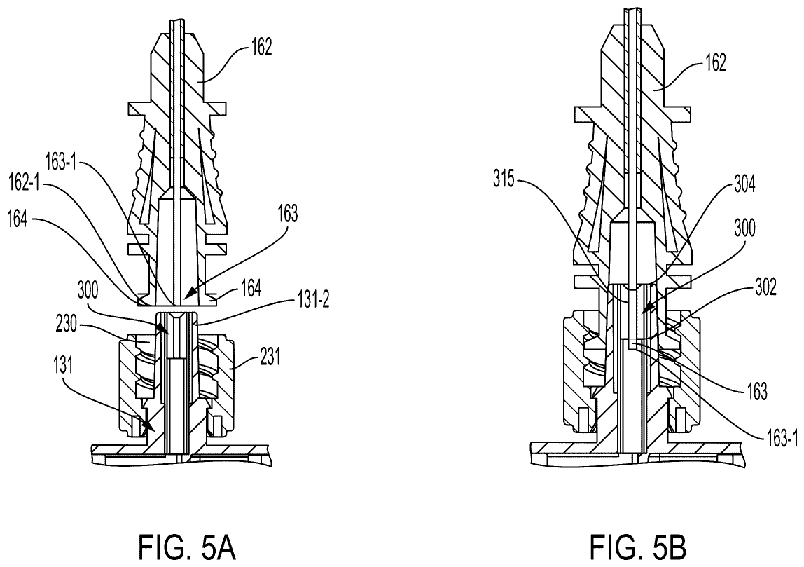

Turning to FIGS. 1-3, the elongate hollow stylet 132 is couplable to the applicator hub 131 via a needle hub 162 to ensure the fluid coupling as described herein. The needle hub 162 includes an interior cannula 163 that fluidly couples the elongate hollow stylet 132 to the output ports 112A-1, 112A-2 of the applicator 110. As shown in FIGS. 2, 5A, and 5B, the interior cannula 163 is positioned within the needle hub 162 such that a proximal end 163-1 of the interior cannula 163 is flush with a proximal end 162-1 of the needle hub 162. The interior cannula 163 is defined by an exterior diameter d_{cannula}.

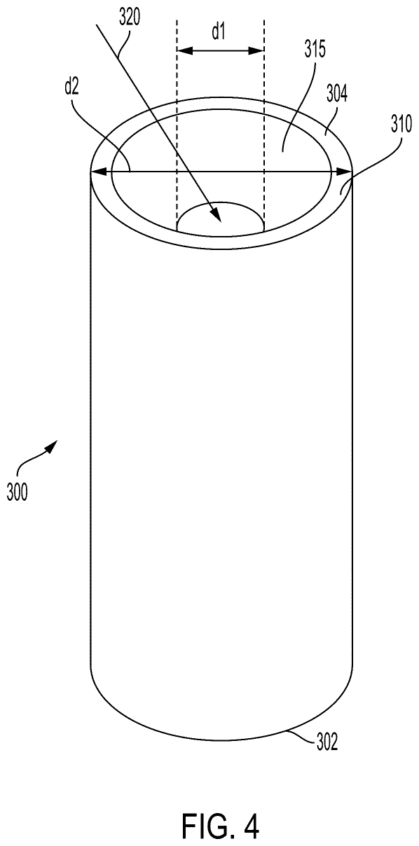

Turning now to FIG. 3, the applicator hub 131 includes a micro seal 300 positioned within a channel 133 extending through the applicator hub 131. As shown in FIG. 4, the micro seal 300 is an elongate and substantially cylindrical tube, although other geometries, shapes, and/or sizes are contemplated and included within the scope of the present disclosure. The micro seal 300 is made from a soft, pliable material, such as a silicone or a silicone-like material, for example. In various instances, the micro seal 300 can be manufactured using a selection of materials, including Buna-N nitrile rubber, for example. The micro seal 300 can be manufactured through 3D printing, such as through microprinting and/or molding, for example.

The micro seal 300 includes a body 310 that has a proximal end 302 and a distal end 304. A central passageway 320 is defined through a central portion of the body 310 of the micro seal 300 to receive at least a portion of the interior cannula 163 of the needle hub 162 therethrough, for example. A dimension of the passageway 320 is defined by an interior diameter d1. The interior diameter dl of the micro seal 300 is smaller, or otherwise less, than the exterior diameter d_{cannula} of the interior cannula 163; however, as shown in FIG. 5B, given the pliable nature of the micro seal 300, the interior cannula 163 is able to be inserted through the passageway 320 upon application of an adequate force. The relationship between the interior diameter d1 of the micro seal 300 and the exterior diameter d_{cannula} of the interior cannula 163 provides a friction-fit between the micro seal 300 and the interior cannula 163 to ensure the interior cannula 163 is closely, or otherwise securely supported by the micro seal 300 and/or otherwise maintain the interior cannula 163 in a desired orientation. The friction-fit formed between the interior diameter d1 of the micro seal 300 and the exterior diameter d_{cannula} of the interior cannula 163 further serves to maintain the integrity of the micro seal 300, prevent leaks, and/or prevent cross-contamination of channels, for example.

The body 310 of the micro seal 300 further is defined by an exterior diameter d2 that is greater than the interior diameter d1. The body 310 includes a tapered portion 315 extending between the exterior diameter d2 and the interior diameter d1. The tapered portion 315 is at least defined on the distal end 304 of the micro seal 300. Such a tapered portion 315 can facilitate alignment between the interior cannula 163 and the passageway 320 as the needle hub 162 is coupled to the applicator hub 131.

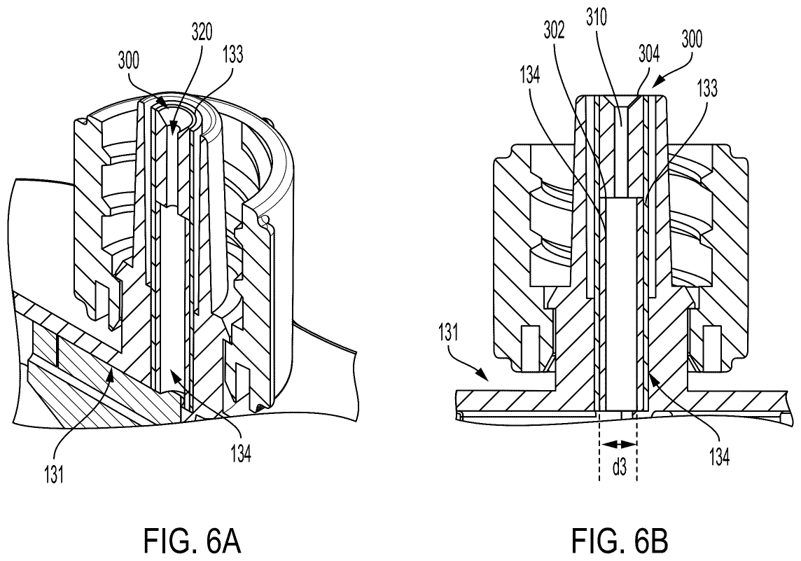

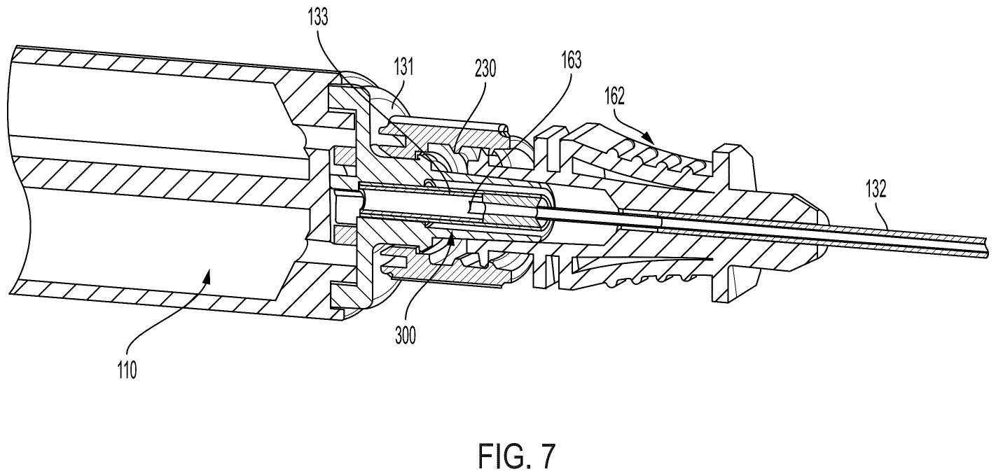

As shown in FIGS. 5A-6B, the micro seal 300 is positioned within a distal portion of the channel 133 of the applicator hub 131 such that the distal end 304 of the micro seal 300 is flush with a distal end 131-2 of the applicator hub 131. The channel 133 is defined by an interior diameter d3 that is smaller, or otherwise less, than the exterior diameter d2 of the micro seal 300; however, given the pliable nature of the micro seal 300, the micro seal 300 is able to be inserted within the channel 133 with the application of an adequate force. The relationship between the exterior diameter d2 of the micro seal 300 and the interior diameter d3 of the channel 133 provides a friction-fit between the micro seal 300 and the channel 133 so as to maintain the micro seal 300 in position within the channel 133.

In various instances, additional support can be provided to maintain the micro seal 300 in an intended position within the channel 133. For example, an adhesive can be applied between the micro seal 300 and the channel 133 to fix, or otherwise secure, the micro seal 300 in place. As shown in FIGS. 6A and 6B, a backstop 134 is positioned adjacent the proximal end 302 of the micro seal 300 within the channel 133 in an effort to maintain the micro seal 300 in position within the channel 133. The backstop 134 can include a tube made of a material that has an increased rigidity in comparison to the pliable nature of the micro seal 300, for example. The backstop 134 can include a plastic and/or a metal material, for example. In various instances, the backstop 134 is integrally formed within the channel 133. In various instances, a length of the backstop 134 corresponds to a length of the micro seal 300. In other instances, the length of the backstop 134 is greater than the length of the micro seal 300. In other instances, the length of the backstop 134 is less than the length of the micro seal 300. Characteristics, such as one or more dimensions, of the backstop 134 can be modified based at least on a material of the backstop 134 and/or a thickness of the backstop 134, for example. In various instances, a UV curing can be applied to at least a portion of the micro seal 300, such as to an outer circumference of the micro seal 300, to prevent unwanted displacement. Alternatively or additionally, the micro seal can be fixed, or otherwise secured, in place through a crimping or a custom manufacturing process, for example.

Referring back to FIGS. 5A and 5B, the proximal end 162-1 of the needle hub 162 includes a lip 164 defined thereon to facilitate connection of the needle hub 162 to the applicator hub 131. One or more threads 230 are defined within the body 231 of the applicator hub 131 to receive at least a portion of the lip 164 as the needle hub 162 is brought into contact with the applicator hub 131. The needle hub 162 is shown detached, or otherwise separate, from the applicator hub 131 in FIG. 5A. As shown in FIG. 5B, as the needle hub 162 is rotated with respect to the applicator hub 131, the one or more threads 230 are engaged by the lip 164, and the needle hub 162 is moved proximally relative to the applicator hub 131.

As the needle hub 162 is coupled to the applicator hub 131 through rotation, the proximal end 163-1 of the interior cannula 163 is inserted into the passageway 320 of the micro seal 300 and the interior cannula 163 passes through the micro seal 300. Once the needle hub 162 is coupled to the applicator hub 131, the interior cannula 163 extends entirely through the micro seal 300. Stated another way, the proximal end 163-1 of the interior cannula 163 is proximal to the proximal end 302 of the micro seal 300 when the needle hub 162 is fully coupled to the applicator hub 131.

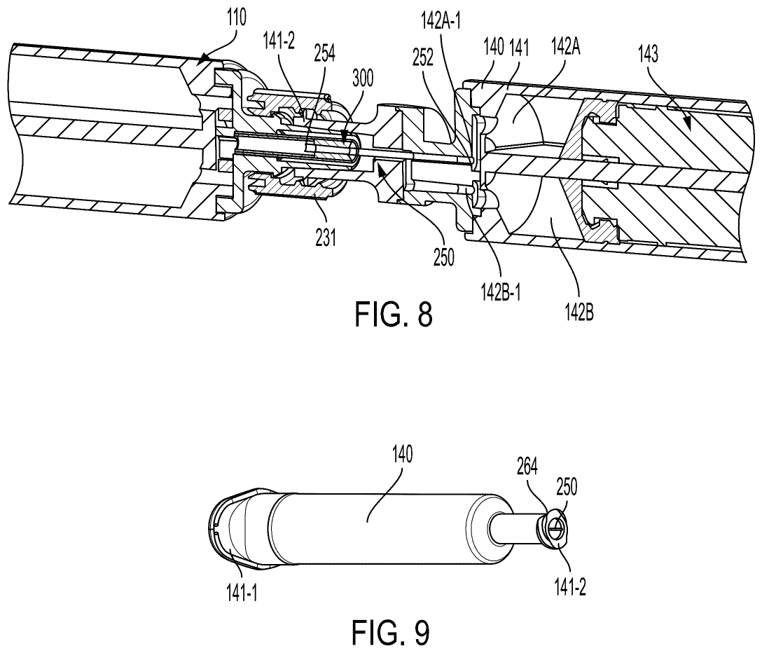

Turning now to FIGS. 8 and 9, a second applicator 140 is couplable to the applicator 110 in lieu of the injection needle assembly 130, for example. The second applicator 140 is couplable to the applicator 110 for the purposes of mixing components stored therein, for example. The second applicator 140 is similar to the applicator 110 in many respects. As such, for the purpose of brevity, the particular details of similar features will not be repeated herein.

The second applicator 140 generally includes a body 141 having a proximal end 141-1 and a distal end 141-2 spaced a distance apart from the proximal end 141-1. The body 141 also defines a pair of syringes 142. The second applicator 140 is configured to separately carry each of a first component of the multi-component material and a second component of the multi-component material.

The pair of syringes 142 includes an actuator 143, a first, or third, component chamber 142A, and a second, or fourth, component chamber 142B. The first component chamber 142A may be, for example, a cylindrical tube that is configured to carry the first component of the multi-component sealant. The first component chamber 142A has a first, or third, output port 142A-1 (e.g., a first component port). The second component chamber 142B also may be, for example, a cylindrical tube that is configured to carry the second component of the multi-component material. The second component chamber 142B has a second, or fourth, output port 142B-1 (e.g., a second component port). In some aspects, the first component chamber 142A and the second component chamber 142B are arranged in a substantially longitudinally parallel arrangement. However, as with the applicator 110, the present disclosure is not limited to such.

The distal end 141-2 of the second applicator 140 includes a lip 264 defined thereon to facilitate connection of the second applicator 140 to the applicator hub 131 of the applicator 110. At least a portion of the lip 264 engages the one or more threads 230 defined within the body 231 of the applicator hub 131 as the second applicator 140 is being coupled to the applicator hub 131.

The second applicator 140 further includes a mixing tube 250 fluidly coupled to the first output port 142A-1 of the first component chamber 142A. The mixing tube 250 includes a proximal end 252 and a distal end 254. The distal end 254 of the mixing tube 250 is flush with the distal end 141-2 of the second applicator 140. Stated another way, the mixing tube 250 does not protrude, or otherwise extend, beyond the distal end 141-2 of the second applicator 140. The mixing tube 250 is defined by an exterior diameter d_{mixing}. The interior diameter d1 of the micro seal 300 is smaller, or otherwise less, than the exterior diameter d_{mixing} of the mixing tube 250; however, as shown in FIG. 8, given the pliable nature of the micro seal 300, the mixing tube 250 is able to be inserted through the passageway 320 upon application of an adequate force. The relationship between the interior diameter d1 of the micro seal 300 and the exterior diameter d_{mixing} of the mixing tube 250 provides a friction-fit between the micro seal 300 and the mixing tube 250 to ensure the mixing tube 250 is closely, or otherwise securely supported by the micro seal 300 and/or otherwise maintain the mixing tube 250 in a desired orientation. The friction-fit formed between the interior diameter dl of the micro seal 300 and the exterior diameter d_{mixing} of the mixing tube 250 further serves to maintain the integrity of the micro seal 300, prevent leaks, and/or prevent cross-contamination of channels, for example.

As the second applicator 140 is coupled to the applicator hub 131 through rotation, the distal end 254 of the mixing tube 250 is inserted into the passageway 320 of the micro seal 300 and the mixing tube 250 passes through the micro seal 300. Once the second applicator is coupled to the applicator hub 131, the mixing tube 250 extends entirely through a length of the micro seal 300. Stated another way, the distal end 254 of the mixing tube 250 is positioned closer to the proximal end 111-1 of the applicator 110 than the proximal end 302 of the micro seal 300 when the second applicator 140 is fully coupled to the applicator hub 131.

A first fluid pathway extends between the first component chamber 142A of the second applicator 140, out of the first output port 142A-1 and through the mixing tube 250. As such, the first fluid pathway extends through the passageway 320 of the micro seal 300. A second fluid pathway extends separately from the first fluid pathway. The second fluid pathway extends between the second component chamber 142B of the second applicator 140 and out of the second output port 142B-1. The second fluid pathway extends on the outside of, or otherwise around, the mixing tube 250 and outside of the micro seal 300. The second fluid pathway does not extend through the passageway 320 of the micro seal 300. As such, the components being mixed within the first component chamber 142A and the second component chamber 142B are kept separate.

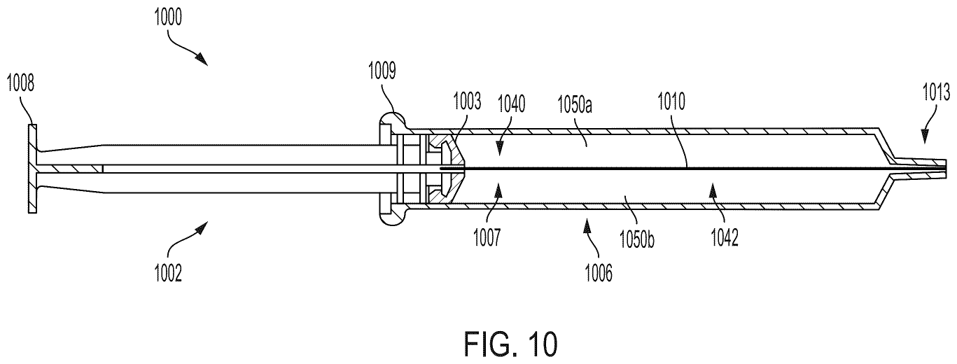

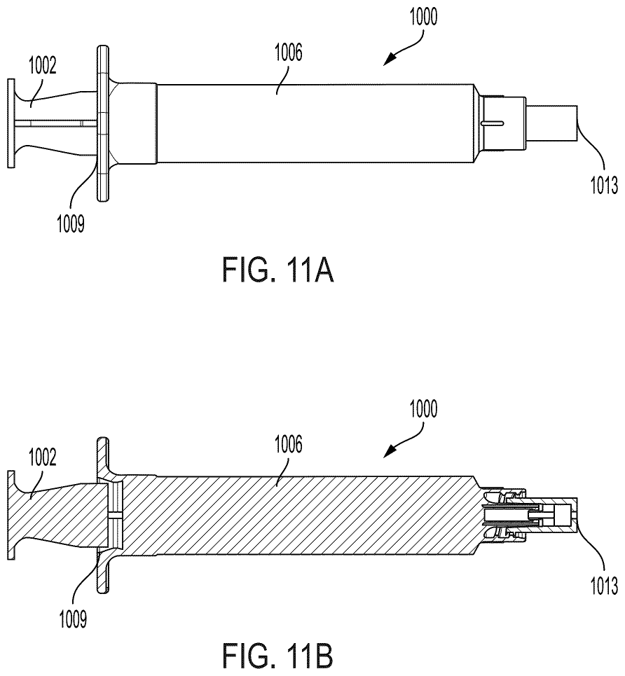

Turning now to FIGS. 10, 11A, and 11B, an illustrative mixing applicator assembly 1000 is depicted. The mixing applicator assembly 1000 may be used for combining two or more constituent materials, as will be described in greater detail herein. In particular, the mixing applicator assembly 1000 may be used to mix or combine two or more constituent components of a hydrogel for use as a radiation spacer. The mixing applicator assembly 1000 generally includes a body 1006 and a plunger 1002 slidably movable within the body 1006. As discussed in greater detail herein, the plunger 1002 can be a bifurcated plunger or two separate, but connected, plunger elements, for example. It is noted that a mixing applicator assembly may include a greater or fewer number of components without departing from the scope of the present disclosure.

The body 1006 of the mixing applicator assembly 1000 may be generally hollow and have plunger-receiving end 1009 at a proximal end for receiving the plungers 1002 and a fluid delivery opening 1013 at a distal end, which may be coupled to a material delivery cannula (not shown, which may include a manifold, delivery needle or the like). In embodiments, the fluid delivery opening 1013 may be axially positioned with a centerline of the body 1006 and/or plunger 1002. However, in other embodiments, the fluid delivery opening 1013 may be offset from a centerline of the barrel body 1006 and/or plunger 1002. Such offset may be useful in preventing the one or more mixing components from blocking the fluid delivery opening 1013. In other embodiments, the fluid delivery opening 1013 may be axially positioned with the centerline of the barrel body 1006 and/or plunger 1002.

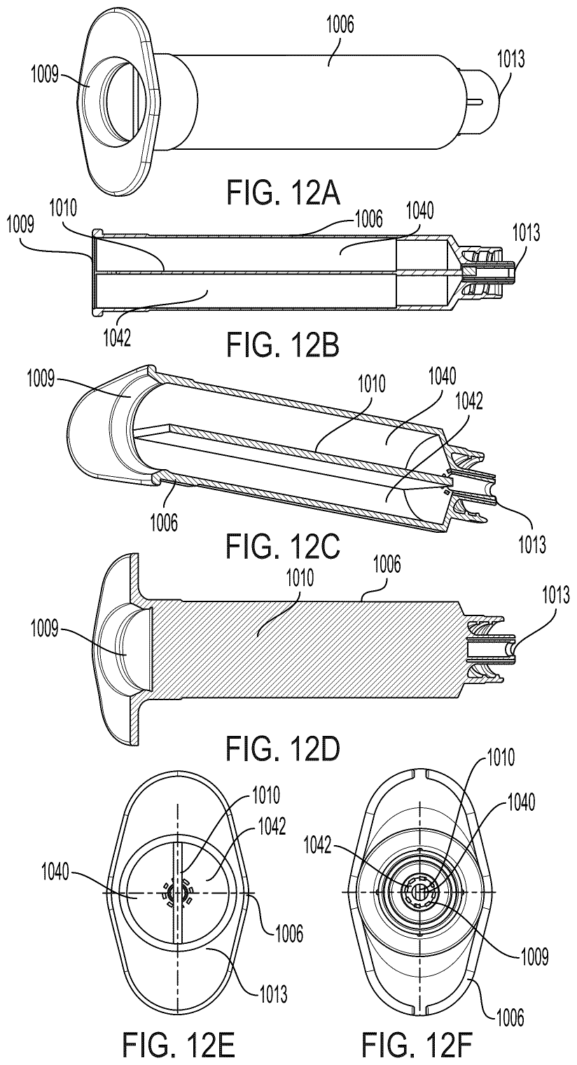

As noted above, the barrel body 1006 is bisected into a plurality of constituent material sections (e.g., lumens 1007). That is, the barrel body 1006 includes a central wall 1010 that divides the body 1006 into a first component chamber 1040 and a second component chamber 1042. The central wall 1010 may extend an entire length of the body 1006 or may only extend a portion of the entire length of the body 1006. In either case, the central wall 1010 maintains a separation of the contents stored within the first and second component chambers 1040, 1042, as described herein. In the embodiment depicted in FIG. 10 and further shown in FIGS. 12A-12F, the central wall 1010 is generally positioned to evenly split the cylindrical body 1006 in half such that a cross section of the body 1006 is two hemispheres (e.g., as shown in FIGS. 12E-12F when viewing the ends of the barrel body 1006 straight on). However, the present disclosure is not limited to such; that is, the central wall 1010 may be located in other positions or other configurations to separate the first and second component chambers 1040, 1042. Further, while only a single central wall 1010 is depicted (forming two component chambers 1040, 1042), the present disclosure is not limited to such. That is, more than one central wall may be used to divide the body 1006 into more than two component chambers (e.g., three component chambers, four component chambers, greater than four component chambers, etc.).

The first component chamber 1040 and the second component chamber 1042 may initially take up the same volume or substantially same volume as depicted. However, in some embodiments, initial volumes may differ from one another. As will be described in greater detail, a first component 1050a may be positioned initially within the first component chamber 1040 and a second component 1050b, different from the first component 1050a, may be positioned within the second component chamber 1042.

The first component 1050a may be a liquid for hydrating the second component 1050b, which may be a powder or particulate. For example, the first component 1050a may be saline, water, deionized water, or the like. As noted above, the second component 1050b may be a powder or particulate material such as but not limited to albumin, polyethylenimine (PEI), an amine containing polyethylene glycol (PEG) or protein, an N-hydroxysuccinimide (NHS) ester component such as PEG-(SS)2, PEG-(SS)4, PEG-(SS)8, PEG-(SG)4, PEG-(SG)8, and/or the like. In some aspects, molecular weights of the PEG components may range from about 2,000 to about 100,000. The powder or particular material may be biodegradable and/or bioabsorbable. As used herein, “biodegradable” and/or “bioabsorbable” refers to a compound that can be absorbed by the surrounding or local tissue of a subject and/or degraded and absorbed by the tissue of the subject.

The powder or particulate material can be composed of various crosslinking substances of varying amounts, designed to allow the hydrogel to last a specific amount of time in situ before degrading. In aspects, the hydrogel components may be selected based on a degradation time that corresponds to the length of anticipated radiation therapy. In aspects the length of anticipated radiation therapy, and thus the targeted time for hydrogel degradation is up to 18 months, for example from the range of about 0 months to about 18 months, including about 1 month, 2 months, 3 months, 4 months, 5 months, 6 months, 7 months, 8 months, 9, months, 10 months, 11 months, 12 months, 13 months, 14 months, 15 months, 16 months, 17 months, and 18 months. It should be understood that the time is merely a rough guide generally used to target appropriate formulation of the hydrogel.

As shown in FIGS. 10-11B, the plunger 1002 is slidably disposed in lumens 1007 of the body 1006 and forms fluidic seals with the body 1006 via seal 1003 at a distal end of the plunger 1002. For example, the seal 1003 may be a rubber or similar material conformable material for forming a fluid-tight seal with the body 1006. At a proximal end of the plunger 1002 may be a pusher flange 1008 for a user to engage for advancing the plunger 1002 along the body 1006.

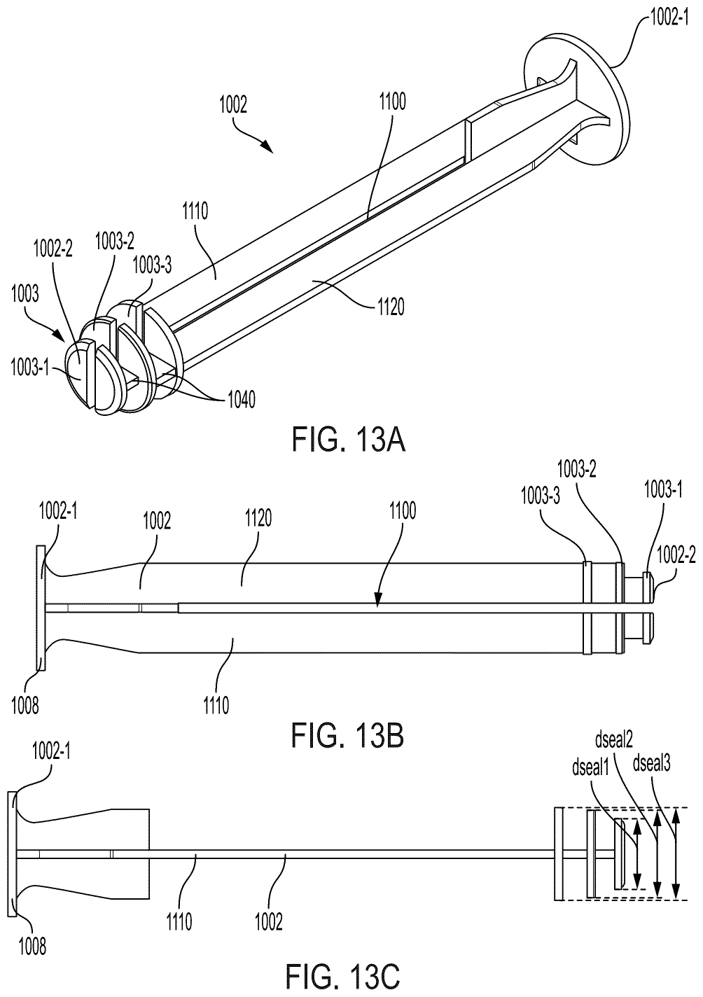

Referring now to FIGS. 13A-13C, in some embodiments, the plunger 1002 may be a bifurcated plunger. That is, the plunger 1002 may be a single piece component with a central channel 1100 defined through a length thereof that is shaped, sized, and located to correspond to the central wall 1010 of the body 1006. Thus, the central channel 1100 defines a first plunger extension 1110 and a second plunger extension 1120, each of which is shaped and sized to correspond to each of the first and second component chambers 140, 142, respectively, such that each plunger extension 1110, 1120, when received within the lumens 1007 of the mixing applicator assembly 1000, seals the respective component chamber 140, 142 as noted hereinabove. Stated another way, as the plunger 1002 is distally advanced through the body 1006 of the mixing applicator assembly 1000, a first plunger extension 1110 is advanced through the first component chamber 1040 and a second plunger extension 1120 is advanced through the second component chamber 1042, for example.

More specifically, a length of the central channel 1100 corresponds to a length of the central wall 1010 defined in the body 1006 of the mixing applicator assembly 1000. In various instances, the length of the central channel 1100 is greater than the length of the central wall 1010 to ensure the plunger 1002 is fully advanced through the mixing applicator assembly 1000, and the entire contents of the component chambers 1040, 1042 are able to be expelled.

The plunger 1002 depicted in FIGS. 13A-13C includes a proximal end 1002-1 and a distal end 1002-2. As discussed with respect to FIGS. 10-11B, one or more seals 1003 are defined on the distal end 1002-2 of the plunger 1002 to form fluidic seals with the body 1006. For example, the seal 1003 may be a rubber or similar material conformable material for forming a fluid-tight seal with the body 1006. As depicted in FIGS. 13A, the seal 1003 formed at the distal end 1002-2 of the plunger 1002 includes three distinct seal components 1003-1, 1003-2, 1003-3. Adjacent seal components 1003-1, 1003-2, 1003-3 can be coupled to one another through connecting members 1140. In embodiments, the connecting members 1140 do not span an entire circumference of the seal components 1003-1, 1003-2, 1003-3.

A first seal component 1003-1 is positioned adjacent to the distal end 1002-2 of the plunger 1002. A second seal component 1003-2 is positioned proximal and adjacent to the first seal component 1003-1. A third seal component 1003-3 is positioned proximal and adjacent to the second seal component 1003-2. As such, the second seal component 1003-2 is positioned intermediate, or otherwise between, the first seal component 1003-1 and the third seal component 1003-3. The first seal component 1003-1 is defined by a first seal diameter dₛₑₐₗ₁. The second seal component 1003-2 is defined by a second seal diameter dₛₑₐₗ₂, which is greater than the first seal diameter dₛₑₐₗ₁. The third seal component 1003-3 is defined by a third seal diameter dₛₑₐₗ₃, which is greater than the first seal diameter dₛₑₐₗ₁ and the second seal diameter dₛₑₐₗ₂. The diameters of the seals taper toward the distal end 1002-2 of the plunger 1002 in accordance with, or to otherwise mimic, a geometry of the body 1006 of the mixing applicator assembly 1000. For example, the third seal component 1003-3 can form a seal with a proximal portion of the body 1006, the second seal component 1003-2 can form a seal with an intermediate portion of the body 1006, and the first seal component 1003-1 can form a seal with a distal portion of the body 1006. As such, a seal can be made along an entire length of the body 1006 of the mixing applicator assembly 1000. As depicted in FIGS. 13A and 13B, the central channel 1100 extends through the seal 1003, including the three distinct seal components 1003-1, 1003-2, 1003-3.

At a proximal end of the plunger 1002 may be a pusher flange 1008 for a user to engage for advancing the plunger 1002 along the body 1006.

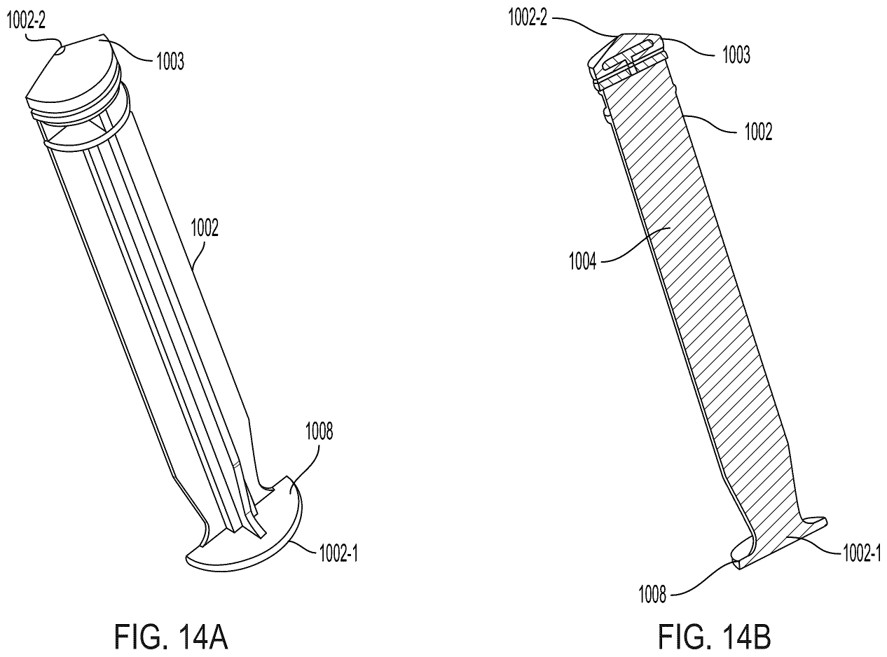

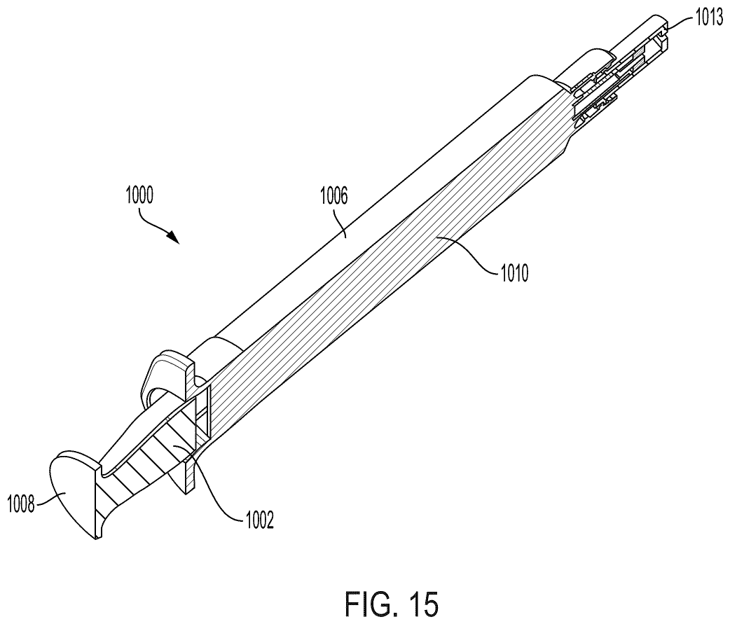

As shown in FIGS. 14A and 14B, in other embodiments, the plunger 1002 may be a “half plunger” or otherwise shaped and sized to correspond to a respective one of the first and second component chambers 1040, 1042 such that each “half plunger” is operable independently of the other. For example, in embodiments where the mixing applicator assembly 1000 is divided into three component chambers by two walls 1010, the plunger 1002 can be a “third plunger” sized to correspond to a respective one of the three component chambers. The plunger 1002 includes a smooth, or otherwise continuous, surface 1004 to slide along each wall 1010 defined in the body 1006 of the mixing applicator assembly 1000. Placement within the respective component chamber 1040, 1042 of the mixing applicator assembly 1000 can be visualized, for example, in FIG. 15. While not depicted, the multiple component plungers, such as the two “half plungers” and/or the three “third plungers,” may be joined together with a mating piece at a proximal end thereof (e.g., a mating piece that extends around and/or is overmolded around the respective pusher flanges 1008 such that the multiple component plungers can be operated together in tandem.

As noted herein, the shape and size of the mixing applicator assembly 1000 is not limited by this disclosure, and may be any shape and/or size. In some embodiments, the mixing applicator assembly 1000 may be shaped and/or sized to ensure a suitable amount of material can be received in each of the component chambers 1040, 1042.

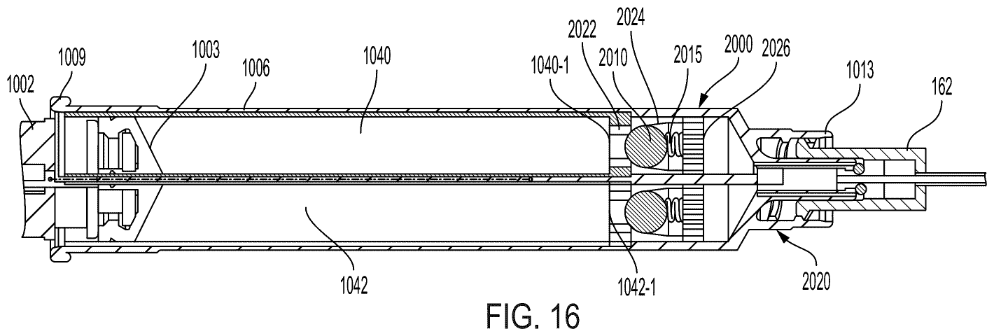

Turning now to FIGS. 16-18, one or more valves 2000 may be integrated within the mixing applicator assembly 1000 to prevent a backflow of materials, for example. The one or more valves 2000 may include a valve closure which is a device configured to be selectively moved to seal off, or otherwise block, a flow path. As depicted in FIGS. 16-18, the value closure may include a ball valve, or the like.

The ball valve 2000 includes a ball or spherical member 2010 and a resilient member 2015, such as a helical spring. The ball valve 2000 is movable between an open configuration to allow fluid to pass freely therethrough and a closed configuration where fluid is prevented from passing, or otherwise flowing, therethrough. The ball valve is positioned distal to the first component chamber 1040 and/or the second component chamber 1042 of the mixing applicator assembly 1000. In various instances, the ball valve 2000 is positioned within a distal end of the first component chamber 1040 and/or the second component chamber 1042.

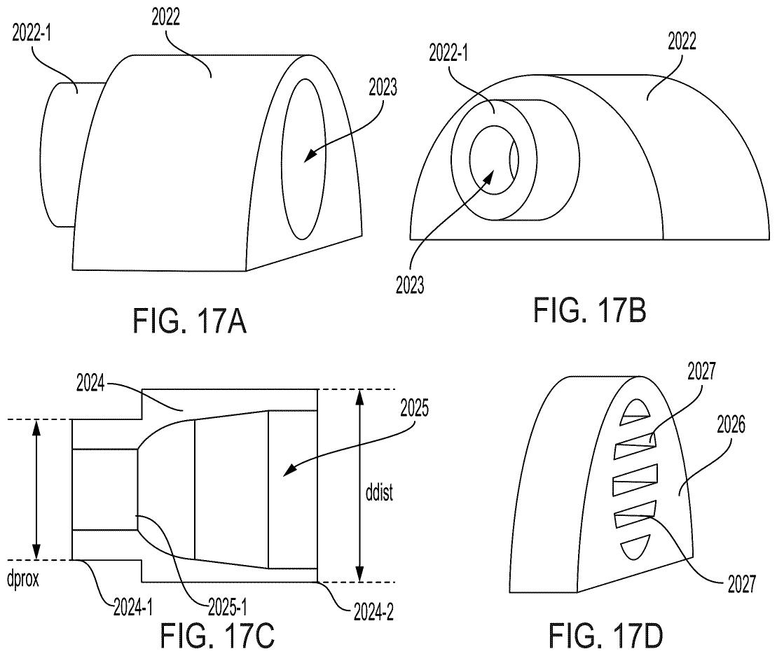

The ball valve 2000 includes a housing 2020 to support the ball member 2010 and the resilient member 2015 therein. The housing 2020 includes a proximal component 2022, an intermediate component 2024, and a distal component 2026. The proximal component 2022 is positioned within the body 1006 of the mixing applicator assembly 1000 such that a proximal end 2022-1 of the proximal component 2022 extends into, or otherwise fluidly mates with, a first output port 1040-1 of the first component chamber 1040. In various instances, as shown in FIGS. 17A and 17B, the proximal end 2022-1 of the proximal component 2022 includes a tubular extension sized to match the dimensions of the first output port 1040-1 of the first component chamber 1040. As such, the tubular extension can be received within the first output port 1040-1 of the first component chamber 1040. The proximal component 2022 further includes a channel 2023 defined therethrough to facilitate a passage of contents between the first component chamber 1040 and the fluid delivery opening 1013 at the distal end of the body 1006.

Referring now to FIGS. 16 and 17C, the intermediate component 2024 of the housing 2020 is positioned distal to the proximal component 2022 of the housing 2020 within the body 1006 of the mixing applicator assembly 1000. A proximal end 2024-1 of the intermediate component 2024 is defined by a diameter dₚᵣₒₓ that permits at least a portion of the proximal end 2024-1 of the intermediate component 2024 to be received within the channel 2023 of the proximal component 2022. A distal end 2024-2 of the intermediate component 2024 is defined by a diameter d_{dist}. The diameter d_{dist} of the distal end 2024-2 of the intermediate component 2024 is greater than the diameter dₚᵣₒₓ of the proximal end 2024-1 of the intermediate component 2024. The diameter d_{dist} of distal end 2024-2 is greater than the diameter of the channel 2023 defined through the proximal component 2022 thereby preventing the distal end 2024-2 of the intermediate component 2024 from passing through the channel 2023 defined through the proximal component 2022.

The intermediate component 2024 further includes a channel 2025 defined and extending therethrough to facilitate a passage of contents between the first component chamber 1040 and the fluid delivery opening 1013 at the distal end of the body 1006 while also supporting the ball member 2010 and the resilient member 2015 therein. The channel 2025 is conical in geometry; however, any suitable geometry is envisioned. The conical nature of the channel 2025 serves to align the ball member 2010 within the channel 2025, for example. The ball member 2010 is movably supported within the channel 2025. In various instances, a proximal end of the resilient member 2015 is fixedly coupled to the ball member 2010. In other instances, the proximal end of the resilient member 2015 rests upon and/or otherwise is in contact with the ball member 2010.

As shown in FIGS. 16 and 17D, the distal component 2026 is positioned within the body 1006 of the mixing applicator assembly 1000 distal to both the intermediate component 2024 and the proximal component 2022. The intermediate component 2024 is positioned between, or otherwise intermediate, the proximal component 2022 and the distal component 2026. The resilient member 2015 is sized so as to extend between the distal component 2026 and the ball member 2010 in its relaxed, or natural, state. In various instances, a distal end of the resilient member 2015 is fixedly coupled to the distal component 2026. In other instances, the distal end of the resilient member 2015 rests upon and/or otherwise contacts the distal component 2026. The distal component 2026 of the housing 2020 includes one or more slits 2027 defined therethrough to facilitate content travel into and out of the first component chamber 1040 and the fluid delivery opening 1013 at the distal end of the body 1006. The slits 2027 can be defined in any geometry and/or frequency within the distal component 2026. For example, the one or more slits 2027 can include a single annular slit.

As shown in FIG. 16, when the ball valve 2000 is in the closed configuration, the ball member 2010 is biased against a base 2025-1 of the channel 2025 by the resilient member 2015 thereby forming a seal between the ball member 2010 and the base 2025-1 of the channel 2025. As such, when the ball valve 2000 is in the closed configuration, a flow path therethrough is blocked and contents are prevented from entering and/or exiting the first component chamber 1040.

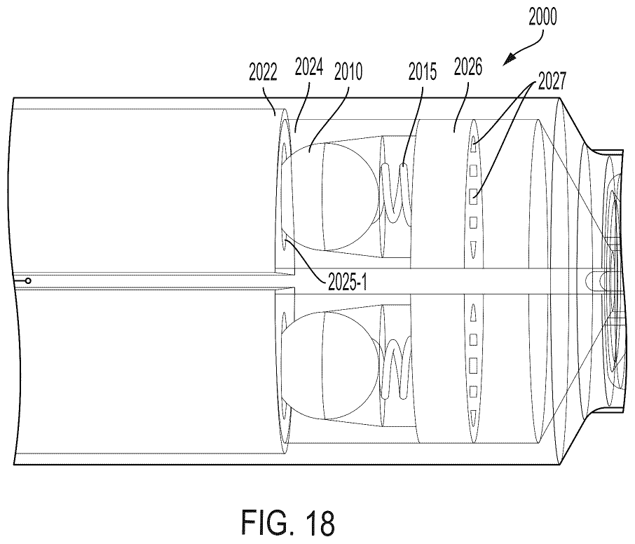

In response to an opposing force, such as the force of a distally-advancing plunger 1002, the ball member 2010 pushes against the resilient member 2015, causing the resilient member 2015 to compress. As shown in FIG. 18, with the resilient member 2015 in a compressed state, the ball member 2010 is distally moved away from the base 2025-1 of the channel 2025. In the absence of the opposing force, such as a proximal retraction of the plunger 1002 and/or a discontinuation of distally-advancing the plunger 1002, the resilient member 2015 returns to its natural state and once again biases the ball member 2010 against the base 2025-1 of the channel 2025. The ball valve 2000 is thus returned to its closed configuration and contents are prevented from entering into and/or exiting the first component chamber 1040.

While the ball valve 2000 is discussed as being positioned distal to the first component chamber 1040, a ball valve 2000 can be alternatively or additionally positioned distal to the second component chamber 1042.

Applicators for use in mixing and/or delivery contents are often coupled to one or more subassemblies to provide connection points for other applicators and/or to provide connection points for other delivery apparatuses (e.g., needles, cannulas, or the like). The disclosure provided herein discusses various improvements on known connection subassemblies by providing dual lumen solutions to maintain a separation between flow pathways of the applicator and/or that allow for quick connection and removal of components. Such subassemblies reduces the complexity and number of components required for the overall device, allowing easier use and function.

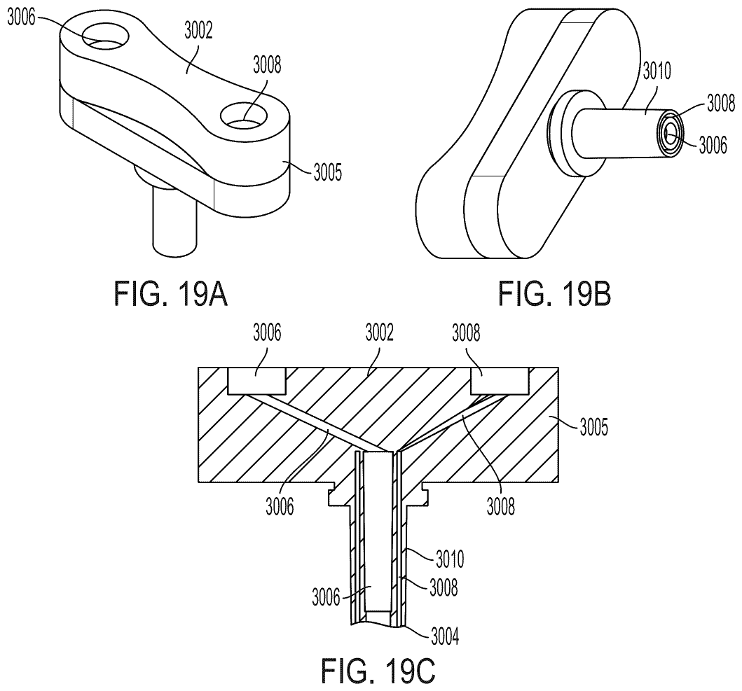

FIGS. 19A-19C depict an applicator hub 3000 for placement on a distal end of a dual-barrel applicator, such as the applicator 110. The applicator hub 3000 includes a body 3005 having a proximal end 3002 and a distal end 3004. The proximal end 3002 includes a first channel 3006 and a second channel 3008 defined therethrough. The first channel 3006 is spaced apart from the second channel 3008. Upon coupling the applicator hub 3000 to the distal end of the dual-barrel applicator, the first channel 3006 is aligned with a first output port of a first component chamber, such as the first component chamber 112A, and the second channel 3008 is aligned with a second output port of a second component chamber, such as the second component chamber 112B, for example.

The distal end 3004 of the applicator hub 3000 includes a tubular projection 3010 extending from the body 3005 of the applicator hub 3000. The first and second channels 3006, 3008 extend separately between the proximal end 3002 of the applicator hub, through the body 3005 of the applicator hub 3000, and through the tubular projection 3010 of the applicator hub 3000. More specifically, as the first and second channels 3006, 3008 converge at the tubular projection 3010 of the applicator hub 3000, the first channel 3006 extends annularly around the second channel 3008. The first channel 3006 and the second channel 3008 are separated within the tubular projection 3010 by an annular wall 3012. As such, contents traveling to and/or from the first component chamber through the first channel 3006 flow through the tubular projection 3010 around, or otherwise outside of, the annular wall 3012. Contents traveling to and/or from the second component chamber through the second channel 3008 flow through the tubular projection 3010 through, or otherwise within, the annular wall 3012. The applicator hub 3000 allows for the contents traveling to and/or from the first and second component chambers to remain separate.

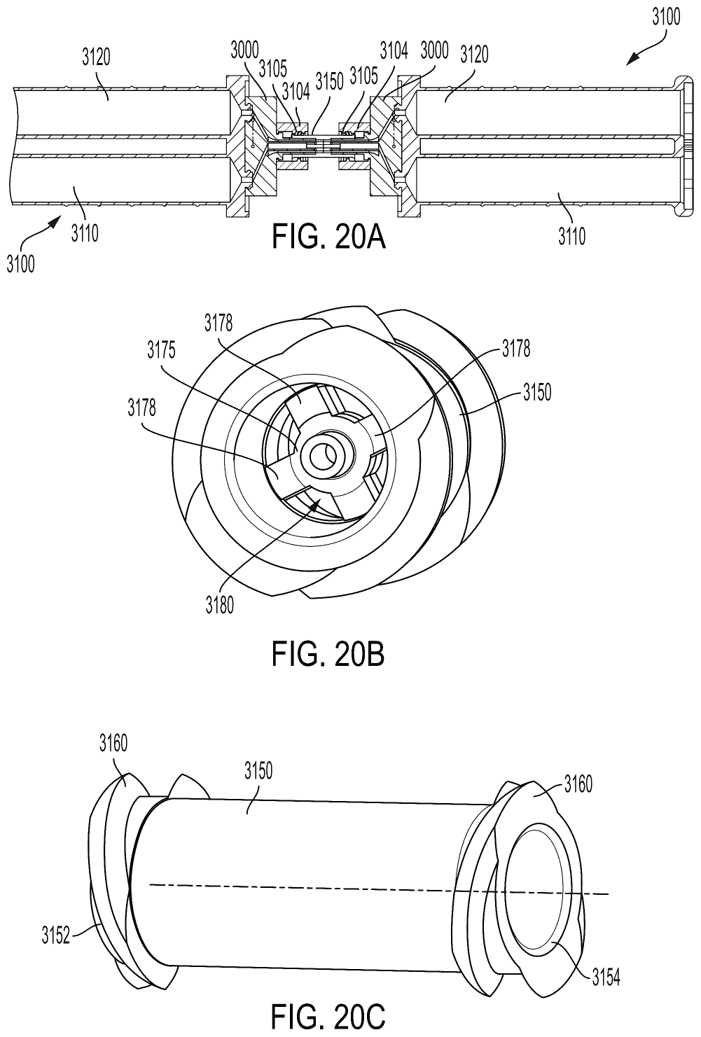

Turning now to FIG. 20A, a first dual barrel applicator 3100 is coupled to a second dual barrel applicator 3100 for mixing contents between respective component chambers. More specifically, the first dual barrel applicator 3100 includes a first component chamber 3110 and a second component chamber 3120. The second dual barrel applicator 3100 similarly include a first component chamber 3110 and a second component chamber 3120. The contents from each of the first component chambers 3110 are intended to be mixed, or otherwise combined, separate from the contents from each of the second component chambers 3120. Similarly, the contents from each of the second component chambers 3120 are intended to be mixed, or otherwise combined, separate from the contents from each of the first component chambers 3110. As such, it is desirable to maintain separation between the contents from the first component chambers 3110 and the second component chambers 3120 during mixing to prevent solidification, or any other adverse reaction.

As depicted in FIG. 20A, the distal ends 3104 of the applicators 3100 include the same male luer lock connection extending from an applicator hub, such as the applicator hub 3000. Such male luer lock connections include one or more threads 3105 defined along an internal surface thereof. As such, without a connector, the distal ends 3104 of the applicator 3100 are not compatible, or are otherwise not supported, for connecting to one another. A connector 3150 is depicted in FIGS. 20A-20C to facilitate connection between the applicators 3100 while also maintaining separation between the contents of the first component chambers 3110 and the second component chambers 3120.

The connector 3150 is substantially cylindrical in geometry. The connector 3150 includes a first end 3152 for connection to the first dual barrel applicator 3100 and a second end 3154 for connection to the second dual barrel applicator 3100. One or more tabs 3160 are defined on an external surface of the first end 3152 of the connector 3150 for engagement with the one or more threads 3105 defined on the internal surface of the distal end 3104 of the first dual barrel applicator 3100. Similarly, one or more tabs 3160 are defined on an external surface of the second end 3154 of the connector 3150 for engagement with the one or more threads 3105 defined on the internal surface of the distal end 3104 of the second dual barrel applicator 3100.

As shown in FIGS. 20A and 20B, the connector 3150 is substantially hollow. As such, the distal ends 3004 of the applicator hubs 3000 extend into each respective side of the connector 3150. However, the distal ends 3004 of the applicator hubs 3000 are prevented from being inserted through an entire length of the connector 3150 by an internal mixing channel 3175 defined through a central portion of the connector 3150. Stated another way, the internal mixing channel 3175 does not extend an entire length of the connector 3150. The internal mixing channel 3175 is aligned with the first channel 3006 of the applicator hub 3000 for receiving the contents of the first component chambers 3110 therethrough. In various instances, at least a portion of the internal mixing channel 3175 is received within the first channel 3006 of the applicator hub 3000. In any event, the contents of the first component chambers 3110 pass between the first channels 3006 of the applicator hubs 3000 by passing inside of, or otherwise through, the internal mixing channel 3175. The contents of the second component chambers 3120 pass between the second channels 3008 of the applicator hubs 3000 by passing outside of, or otherwise around, the internal mixing channel 3175.

The internal mixing channel 3175 is fixed, or otherwise supported, within a central portion of connector 3150 by one or more projections 3178 extending from an internal sidewall of the connector 3150. The one or more projections 3178 are separate and distinct from one another such that contents from the second component chambers 3120 can pass through the openings 3180 defined therebetween.

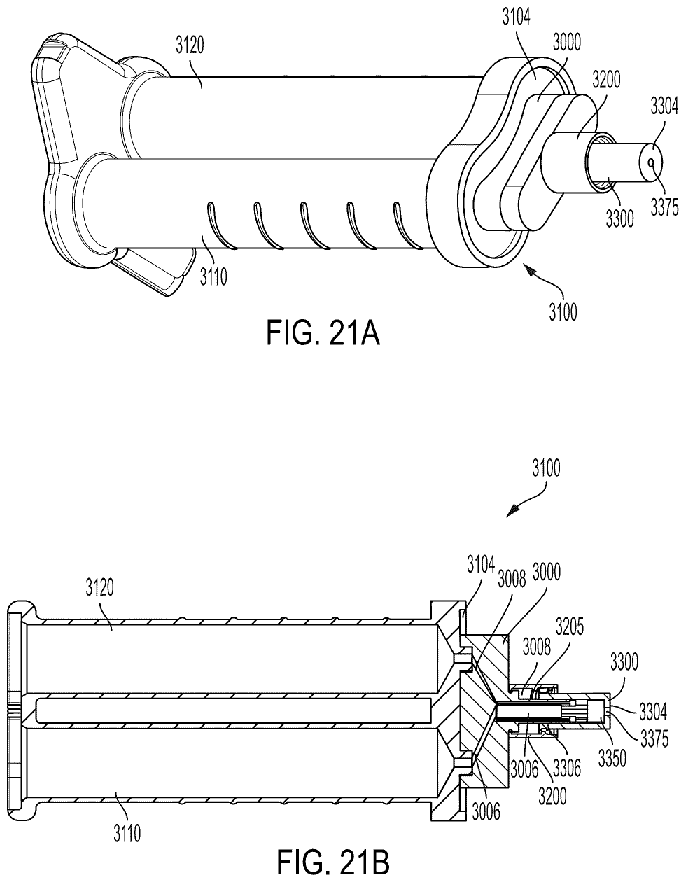

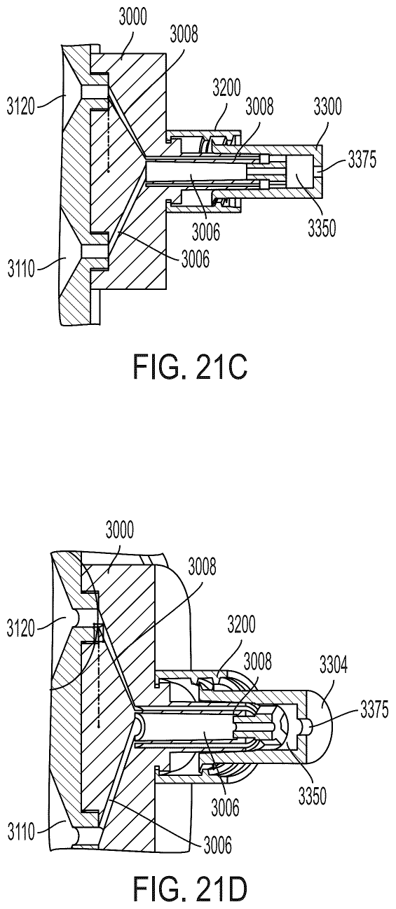

FIGS. 21A-21D depict the dual barrel applicator 3100. The dual barrel applicator 3100 depicted in FIGS. 21A-21D is intended to mix, or otherwise combine, the contents of the first component chamber 3110 and the second component chamber 3120 of a single dual barrel applicator 3100. However, the respective contents for the first component chamber 3110 and the second component chamber 3120 are not intended to be mixed, or otherwise combined, until just before the contents are dispensed from the applicator 3100.

An applicator hub, such as the applicator hub 3000, is coupled to a distal end 3104 of the applicator 3100. Furthermore, a male luer lock connector 3200 extends from, or is otherwise coupled to, the applicator hub 3000. The luer lock connector 3200 includes one or more threads 3205 defined along an internal surface thereof.

A mixing connector 3300 is threadably coupled to the male luer lock connector 3200. More specifically, a proximal end of the mixing connector 3300 includes one or more tabs 3306 defined on an external surface thereof for engagement with the one or more threads 3205 defined on the internal surface of the luer lock connector 3200.

The mixing connector 3300 is substantially hollow. As such, at least a portion of the applicator hub 3000 extends into, or is otherwise received by, the mixing connector 3300. A space defined between the distal end of the applicator hub 3000 and a distal end 3304 of the mixing connector 3300 serves as a mixing chamber 3350. As contents from the first component chamber 3110 exit the first channel 3006 of the applicator hub 3000, the contents are received within the mixing chamber 3350. Similarly, as contents from the second component chamber 3120 exit the second channel 3008 of the applicator hub 3000, the contents are received within the mixing chamber 3350. As such, the contents from both component chambers 3110, 3120 can be mixed, or otherwise combined, within the mixing chamber 3350. The mixing connector 3300 further includes a single aperture 3375 defined through the distal end 3304 for dispensing the mixed contents therethrough. Alternatively, a needle assembly can be inserted through the aperture 3375 for dispensing the mixed contents therethrough.

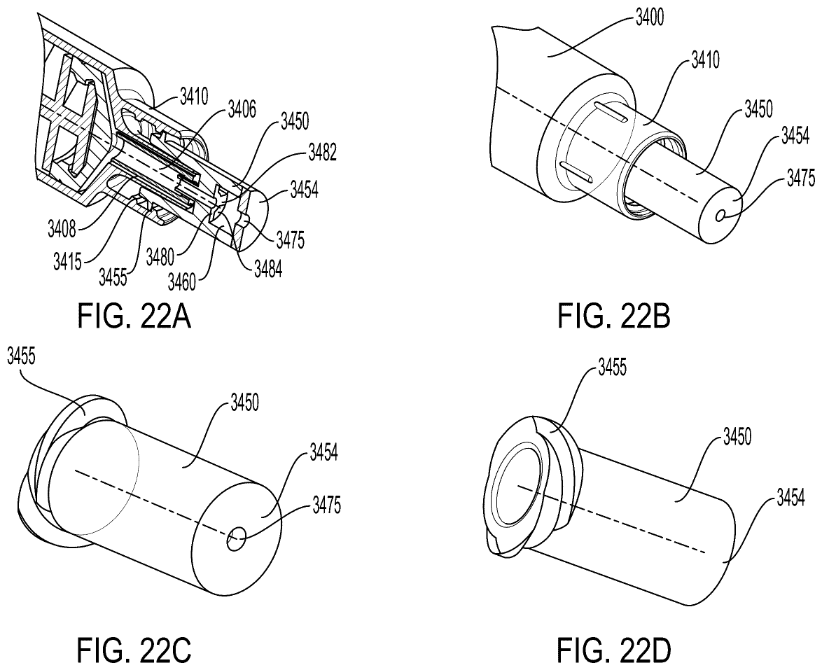

FIGS. 22A-22D depict a mixing connector 3450 coupled to a distal end of an applicator 3400. A distal end of the applicator 3400 includes a male luer lock connector 3410 coupled to, or otherwise defined thereon. More specifically, the luer lock connector 3410 includes one or more threads 3415 defined along an internal surface thereof. A mixing connector 3450 is threadably coupled to the male luer lock connector 3410. A proximal end of the mixing connector 3450 includes one or more tabs 3455 defined on an external surface thereof for engagement with the one or more threads 3415 defined on the luer lock connector 3410.

The mixing connector 3450 is substantially hollow. As such, at least a portion of the applicator 3400 extends into, or is otherwise received by, the mixing connector 3450. A space defined between the distal end of the applicator 3400 and a distal end 3454 of the mixing connector 3450 serves as a mixing chamber 3460. As first contents from the applicator 3400 exit a first channel 3406, the first contents are received within the mixing chamber 3460. Similarly, as second contents exit a second channel 3408, the second contents are received within the mixing chamber 3460. As such, the first and second contents can be mixed, or otherwise combined, within the mixing chamber 3460. The mixing connector 3450 further includes a single aperture 3475 defined through the distal end 3454 for dispensing the mixed contents therethrough. Alternatively, a needle assembly can be inserted through the aperture 3475 for dispensing the mixed contents therethrough.

As depicted in FIG. 22A, the applicator 3400 is prevented from being inserted through an entire length of the mixing connector 3450 by an internal mixing channel 3480 defined though a portion of the mixing connector 3450. Stated another way, the internal mixing channel 3480 does not extend an entire length of the mixing connector 3450. The internal mixing channel 3480 is aligned with the first channel 3406 for receiving the first contents therethrough. In various instances, at least a portion of the internal mixing channel 3480 is received within the first channel 3406. In any event, the first contents pass through the first channel 3406 by passing inside of, or otherwise through, the internal mixing channel 3480. The second contents pass between the second channel 3408 by passing outside of, or otherwise around, the internal mixing channel 3480.

The internal mixing channel 3480 is fixed, or otherwise supported, within a central portion of the mixing connector 3450 by one or more projections 3482 extending from an internal sidewall of the connector 3450. The one or more projections 3482 are separate and distinct from one another such that the second contents can pass through the openings 3484 defined therebetween.

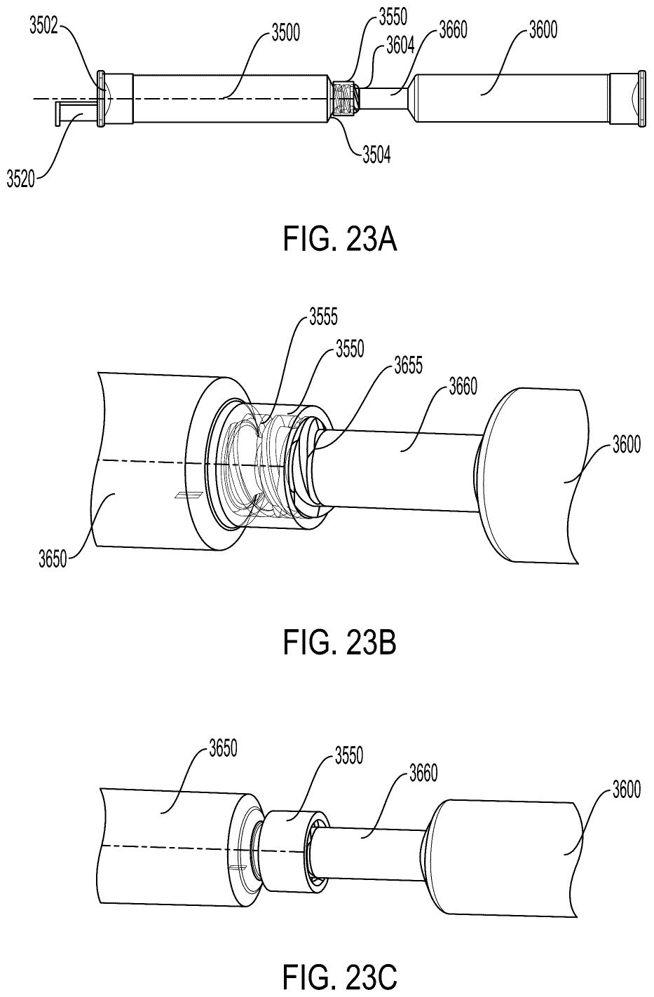

FIGS. 23A-23C depict a mixing assembly including a first applicator 3500 and a second applicator 3600. The first applicator 3500 is similar in many respects to the second applicator 3600. The first applicator 3500 includes one or more component chambers for storing and/or mixing contents therein. The first applicator 3500 includes a proximal end 3502 and a distal end 3504. The proximal end 3502 includes one or more openings for receiving one or more plungers 3520 therethrough. In various instances, a distal end 3604 of the second applicator 3600 includes an elongated tube 3600. Such an elongated tube 3600 can provide a region for a user to grasp, or otherwise align, the second applicator 3600 in a desirable position relative to the first applicator 3500.

The distal end 3504 of the first applicator 3500 includes a male luer lock connector 3550 defined thereon, or otherwise coupled thereto. More specifically, the male luer lock connector 3550 includes one or more threads 3555 defined on an internal surface thereof. The distal end 3604 of the second applicator 3600 includes one or more tabs 3655 defined on an external surface thereof for engaging the one or more threads 3555 of the male luer lock connector 3550. The male luer lock connector 3550 can be fixed to the distal end 3504 of the first applicator 3500 or rotatable relative to the distal end 3504 of the first applicator. Stated another way, as the second applicator 3600 having the one or more tabs 3655 defined thereon is brought into contact with the male luer lock connector 3550, the male luer lock connector 3550 can be rotated, or otherwise spun, to easily engage the tabs 3655 of the second applicator 3600. As the one or more tabs 3655 of the second applicator 3600 are engaged by the one or more threads 3555 of the male luer lock connector 3550, the first applicator 3500 is secured to the second applicator 3600.

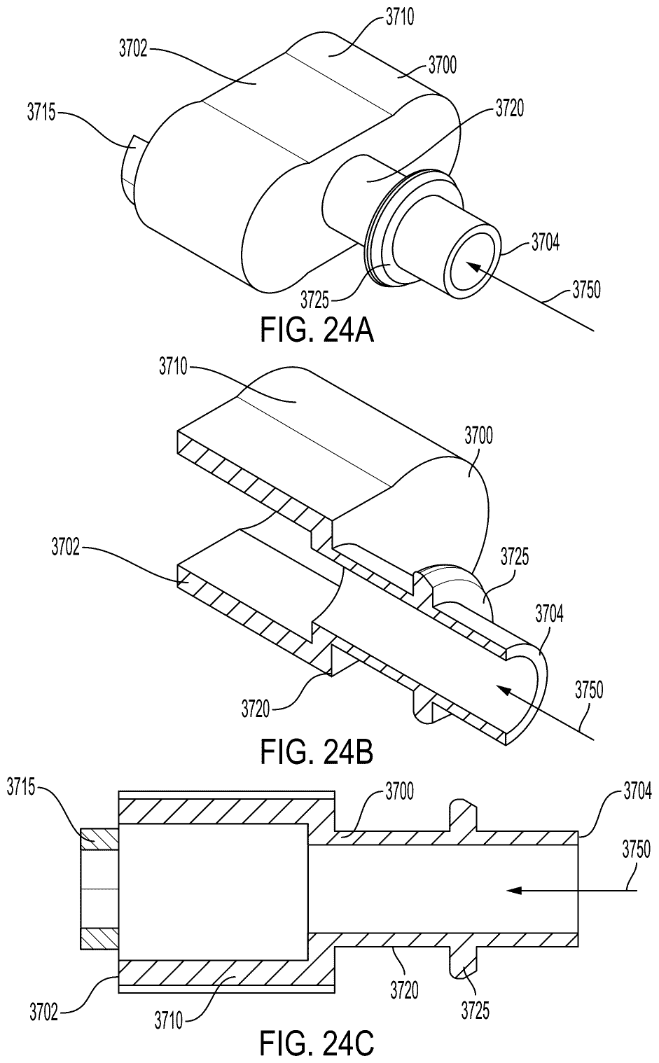

FIGS. 24A-24C depict an applicator hub 3700 for coupling by a press-fit connection to a distal end of an applicator. The applicator hub 3700 includes a body 3710 having a proximal end 3702 and a distal end 3704. One or more tabs, or extensions, 3715 extend proximally from the proximal end 3702 to engage with a corresponding recess defined in the mating applicator. The body 3710 is sized and/or shaped to receive at least a portion of the applicator therein. An elongate tube 3720 extends distally from the body 3710. A channel 3750 is defined through the elongate tube 3720 and the body 3710 of the applicator hub 3700 for fluidly coupling any fluid pathways within the applicator therethrough. In various instances, as depicted in FIGS. 24A-24C, an annular projection 3725 is defined along a portion of an outer surface of the elongate tube 3750. Such annular projection 3725 can assist in positioning, or otherwise handling, of the applicator hub 3700, for example. The annular projection 3725 can further assist in engaging, or otherwise coupling, with an additional connector, such as a male luer lock connector, for example.

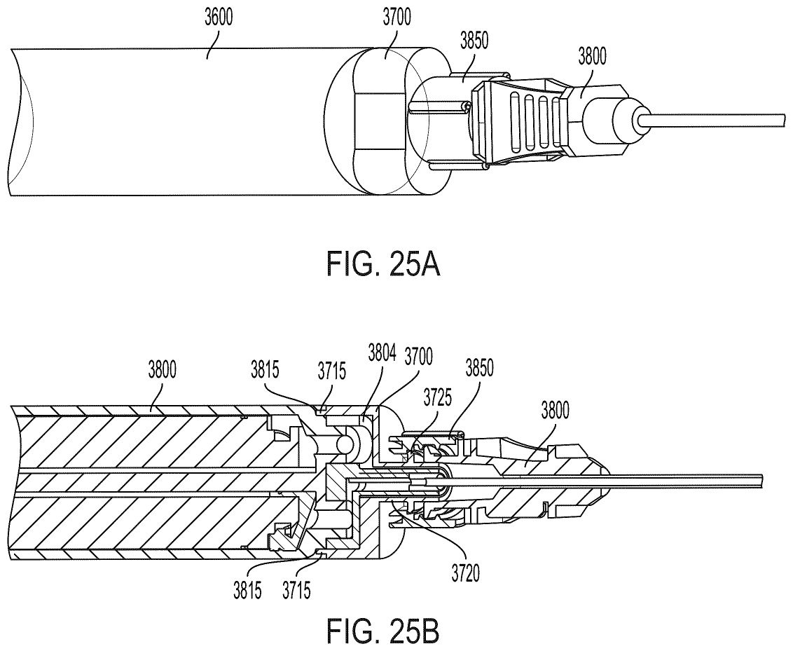

The applicator hub 3700 is shown connected, press-fit, or otherwise coupled to a distal end 3804 of an applicator 3800 in FIGS. 25A and 25B. As discussed herein, at least a portion of the applicator 3800 is received within the applicator hub 3700. The one or more tabs, or extensions, 3715 of the applicator hub 3700 engage with corresponding recesses 3815 defined within the applicator 3800 to secure the applicator hub 3700 to the distal end 3804 of the applicator 3800. Additional delivery connections, such as a needle connector 3900, can be coupled to the distal end 3704 of the applicator hub 3700. The needle connector 3900 can be secured to the applicator hub 3700 using a luer lock connection 3850, for example. In such instances, the annular projection 3725 of the applicator hub 3700 can engage a portion of the luer lock connection 3850.

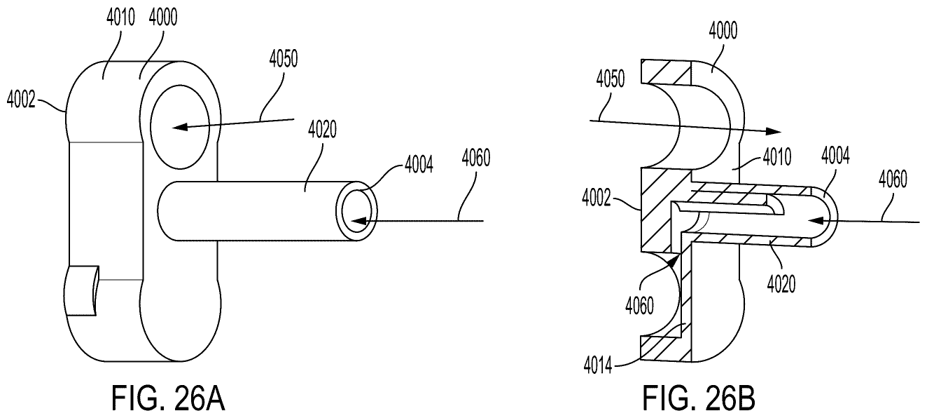

FIGS. 26A and 26B depict an applicator hub 4000 sized to be press-fit to a distal end of a dual-barrel applicator. The applicator hub 4000 includes a body 4010 having a proximal end 4002 and a distal end 4004. The body 4010 includes an elongate tube 4020 extending distally therefrom. The body 4010 further includes a first channel 4050 and a second channel 4060 defined therethrough. The first channel 4050 is spaced apart from the second channel 4060, such that contents flowing through the first channel 4050 remain separate from contents flowing through the second channel 4060.

When the applicator hub 4000 is secured to the dual-barrel applicator, the first channel 4050 is aligned with a first output port of a first component chamber of the applicator while the second channel 4060 is aligned with a second output port of a second component chamber of the applicator. In various instances, at least a portion of the first output port extends into, or through, the first channel 4050, and at least a portion of the second output port extends into, or through, the second channel 4060. The first channel 4050 extends directly through the body 4010 of the applicator hub 4000. A fluid pathway through the second channel 4060 abuts a distal wall 4014 of the body 4010 and redirects through the elongate tube 4020. As such, the contents from the first component chamber remain separate from the contents from the second component chamber.

It should now be understood that the present disclosure relates to various mixing syringe assemblies and methods of mixing constituent materials with mixing syringe assemblies. The various embodiments provided herein may provide ready to use or easily assembled syringe assemblies for easily mixing components. Moreover, embodiments as provided herein may assist in maintaining sterility and/or constituent material integrity, while improving ease of mixing and delivery. The advantages of the present disclosure include that the disclosed devices and assemblies allow for the evolution from a standard syringe capable of only delivering one solution per injection (or a pre mixed solution) to allowing solution constituents to be separated up until delivery. Further advantages of this disclosure include a reduction in the required number of connections pieces down to a single piece instead of several components. The present disclosure further reduces overall device complexity, reduces overall cost, and enhances a user experience by simplifying the connection components between an applicator and any desired connection components such as a second applicator, an applicator hub, a needle hub, and/or the like.

### Examples

Example 1—A system for mixing and delivering content is disclosed. The system includes an applicator, a needle assembly, and a micro seal. The applicator includes two component chambers separate from one another, each component chamber including at least one output port on a distal end thereof and an applicator hub disposed on a distal end of the applicator adjacent to the at least one output port of each component chamber. The needle assembly includes an elongate hollow stylet having a plurality of lumens therein and a needle hub having an interior cannula fluidly coupled to the elongate hollow stylet. The micro seal is disposed within the applicator hub. The micro seal is configured to receive the interior cannula of the needle hub to establish a fluid coupling between each of the two component chambers with a respective one of the lumens within the elongate hollow stylet.

Example 2—The system of Example 1, wherein the micro seal includes a proximal end and a distal end, and wherein the distal end of the micro seal is positioned flush with a distal end of the applicator hub.

Example 3—The system of any one of the preceding Examples, further including a backstop positioned adjacent the proximal end of the micro seal within the applicator hub, wherein the backstop maintains the micro seal in a desired orientation.

Example 4—The system of any one of the preceding Examples, wherein the micro seal includes a body, wherein a passageway is defined through the body, wherein the passageway is defined by an interior diameter, wherein the body is further defined by an exterior diameter, and wherein the exterior diameter is greater than the interior diameter.

Example 5—The system of any one of the preceding Examples, wherein the interior cannula includes an exterior diameter, and wherein the exterior diameter of the interior cannula is greater than the interior diameter of the passageway.

Example 6—The system of any one of the preceding Examples, wherein the micro seal is positioned in a channel defined within the applicator hub, wherein the channel includes an interior diameter, and wherein the interior diameter of the channel is less than the exterior diameter of the micro seal.

Example 7—The system of any one of the preceding Examples, wherein the interior cannula includes a proximal end, wherein the proximal end of the interior cannula is positioned proximal to the proximal end of the micro seal when the needle hub is coupled to the applicator hub.

Example 8—The system of any one of the preceding Examples, wherein the body of the micro seal includes a tapered portion extending between the exterior diameter and the interior diameter on a distal end of the micro seal.

Example 9—The system of any one of the preceding Examples, wherein the applicator hub includes a tab, wherein the applicator includes a recess defined therein, wherein the tab of the applicator hub is press-fit into the recess to secure the applicator hub to the distal end of the applicator.

Example 10—A system for mixing and delivering content is disclosed. The system includes a first applicator, a second applicator, and a micro seal. The first applicator includes a first component chamber including a first output port defined on a distal end thereof, a second component chamber separate from the first component chamber, the second component chamber including a second output port defined on a distal end thereof, and an applicator hub disposed on a distal end of the first applicator adjacent to the first output port and the second output port. The second applicator includes a third component chamber including a third output port defined on a distal end thereof, a fourth component chamber separate from the third component chamber, the fourth component chamber including a fourth output port defined on a distal end thereof, and a mixing tube fluidly coupled to the third output port of the third component chamber. The micro seal is disposed within the applicator hub of the first applicator, wherein the micro seal is configured to receive at least a portion of the mixing tube of the second applicator to establish a first fluid pathway between the first component chamber of the first applicator and the third component chamber of the second applicator separate from a second fluid pathway between the second component chamber of the first applicator and the fourth component chamber of the second applicator.

Example 11—The system of any one of the preceding Examples, wherein the micro seal includes a body, wherein a passageway is defined through the body, wherein the passageway is defined by an interior diameter, wherein mixing tube includes an exterior diameter, and wherein the exterior diameter of the mixing tube is greater than the interior diameter of the passageway.

Example 12—The system of any one of the preceding Examples, wherein the first fluid pathway extends between the first component chamber and the third component chamber, the first fluid pathway extending through the first output port, through the mixing tube and the micro seal, and through the third output port.

Example 13—The system of any one of the preceding Examples, wherein the second fluid pathway extends between the second component chamber and the fourth component chamber, the second fluid pathway extending through the second output port, around the mixing tube and the micro seal, and through the fourth output port.

Example 14—The system of any one of the preceding Examples, wherein the first applicator includes a central wall extending a length of the first applicator to separate the first component chamber from the second component chamber.

Example 15—The system of any one of the preceding Examples, wherein the first applicator further includes a plunger movable through the first component chamber and the second component chamber, wherein the plunger includes a central channel defined therein for slidably receiving the central wall therethrough.

Example 16—The system of any one of the preceding Examples, wherein the first applicator includes a first plunger movable through the first component chamber and a second plunger movable through the second component chamber.

Example 17—A system for mixing and delivering content is disclosed. The system includes a first applicator, a second applicator, a needle assembly, and a micro seal. The first applicator includes a first component chamber including a first output port defined on a distal end thereof, a second component chamber separate from the first component chamber, the second component chamber including a second output port defined on a distal end thereof, and an applicator hub disposed on a distal end of the first applicator adjacent to the first output port and the second output port. The second applicator is removably coupled to the first applicator, wherein the second applicator includes a third component chamber including a third output port defined on a distal end thereof, a fourth component chamber separate from the third component chamber, the fourth component chamber including a fourth output port defined on a distal end thereof, and a mixing tube fluidly coupled to the third output port of the third component chamber. The needle assembly includes an elongate hollow stylet having a plurality of lumens therein and a needle hub having an interior cannula fluidly coupled to the elongate hollow stylet, wherein the needle assembly is removably coupled to the distal end of the first applicator in lieu of the second applicator. The micro seal is disposed within the applicator hub of the first applicator, wherein the micro seal is configured to receive at least a portion of the mixing tube of the second applicator when the second applicator is coupled to the first applicator to establish a first fluid pathway between the first component chamber of the first applicator and the third component chamber of the second applicator separate from a second fluid pathway between the second component chamber of the first applicator and the fourth component chamber of the second applicator, and wherein the micro seal is configured to receive the interior cannula of the needle hub when the needle assembly is coupled to the first applicator to establish a fluid coupling between the first component chamber and the second component chamber with a respective one of the lumens within the elongate hollow stylet.

Example 18—The system of any one of the preceding Examples, wherein the first applicator includes a ball valve positioned adjacent the distal end of the first component chamber.

Example 19—The system of any one of the preceding Examples, wherein the first applicator includes a first male luer lock connector defined on the distal end of the first applicator, wherein the second applicator includes a second male luer lock connector defined on the distal end of the second applicator, wherein a connector couples the distal end of the first applicator to the distal end of the second applicator, and wherein the connector includes one or more tabs defined on a first end thereof for engaging the first male luer lock connection and one or more tabs defined on a second end thereof for engaging the second male luer lock connection.

Example 20—The system of any one of the preceding Examples, wherein the connector further includes an internal mixing chamber defined therethrough, wherein the internal mixing chamber receives contents of the first component chamber therethrough, and wherein contents of the second component chamber passes around the internal mixing chamber.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A system for mixing and delivering content, the system comprising:
an applicator comprising:
two component chambers separate from one another, each component chamber comprising at least one output port on a distal end thereof; and
an applicator hub disposed on a distal end of the applicator adjacent to the at least one output port of each component chamber; and
a needle assembly comprising an elongate hollow stylet having a plurality of lumens therein and a needle hub having an interior cannula fluidly coupled to the elongate hollow stylet; and
a micro seal disposed within the applicator hub, the micro seal configured to receive the interior cannula of the needle hub to establish a fluid coupling between each of the two component chambers with a respective one of the lumens within the elongate hollow stylet.

2. The system of claim 1, wherein the micro seal comprises a proximal end and a distal end, and wherein the distal end of the micro seal is positioned flush with a distal end of the applicator hub.

3. The system of claim 2, further comprising a backstop positioned adjacent the proximal end of the micro seal within the applicator hub, wherein the backstop maintains the micro seal in a desired orientation.

4. The system of claim 1, wherein the micro seal comprises a body, wherein a passageway is defined through the body, wherein the passageway is defined by an interior diameter, wherein the body is further defined by an exterior diameter, and wherein the exterior diameter is greater than the interior diameter.

5. The system of claim 4, wherein the interior cannula comprises an exterior diameter, and wherein the exterior diameter of the interior cannula is greater than the interior diameter of the passageway.

6. The system of claim 5, wherein the micro seal is positioned in a channel defined within the applicator hub, wherein the channel comprises an interior diameter, and wherein the interior diameter of the channel is less than the exterior diameter of the micro seal.

7. The system of claim 2, wherein the interior cannula comprises a proximal end, wherein the proximal end of the interior cannula is positioned proximal to the proximal end of the micro seal when the needle hub is coupled to the applicator hub.

8. The system of claim 4, wherein the body of the micro seal comprises a tapered portion extending between the exterior diameter and the interior diameter on a distal end of the micro seal.

9. The system of claim 1, wherein the applicator hub comprises a tab, wherein the applicator comprises a recess defined therein, wherein the tab of the applicator hub is press-fit into the recess to secure the applicator hub to the distal end of the applicator.

10. A system for mixing and delivering content, the system comprising:
a first applicator comprising:
a first component chamber comprising a first output port defined on a distal end thereof;
a second component chamber separate from the first component chamber, the second component chamber comprising a second output port defined on a distal end thereof;
an applicator hub disposed on a distal end of the first applicator adjacent to the first output port and the second output port;
a second applicator comprising:
a third component chamber comprising a third output port defined on a distal end thereof;
a fourth component chamber separate from the third component chamber, the fourth component chamber comprising a fourth output port defined on a distal end thereof; and
a mixing tube fluidly coupled to the third output port of the third component chamber; and
a micro seal disposed within the applicator hub of the first applicator, the micro seal configured to receive at least a portion of the mixing tube of the second applicator to establish a first fluid pathway between the first component chamber of the first applicator and the third component chamber of the second applicator separate from a second fluid pathway between the second component chamber of the first applicator and the fourth component chamber of the second applicator.

11. The system of claim 10, wherein the micro seal comprises a body, wherein a passageway is defined through the body, wherein the passageway is defined by an interior diameter, wherein mixing tube comprises an exterior diameter, and wherein the exterior diameter of the mixing tube is greater than the interior diameter of the passageway.

12. The system of claim 10, wherein the first fluid pathway extends between the first component chamber and the third component chamber, the first fluid pathway extending through the first output port, through the mixing tube and the micro seal, and through the third output port.

13. The system of claim 12, wherein the second fluid pathway extends between the second component chamber and the fourth component chamber, the second fluid pathway extending through the second output port, around the mixing tube and the micro seal, and through the fourth output port.

14. The system of claim 10, wherein the first applicator comprises a central wall extending a length of the first applicator to separate the first component chamber from the second component chamber.

15. The system of claim 14, wherein the first applicator further comprises a plunger movable through the first component chamber and the second component chamber, wherein the plunger comprises a central channel defined therein for slidably receiving the central wall therethrough.

16. The system of claim 14, wherein the first applicator comprises a first plunger movable through the first component chamber and a second plunger movable through the second component chamber.

17. A system for mixing and delivering content, the system comprising:
a first applicator comprising:
a first component chamber comprising a first output port defined on a distal end thereof;
a second component chamber separate from the first component chamber, the second component chamber comprising a second output port defined on a distal end thereof; and
an applicator hub disposed on a distal end of the first applicator adjacent to the first output port and the second output port;
a second applicator removably coupled to the first applicator, the second applicator comprising:
a third component chamber comprising a third output port defined on a distal end thereof;
a fourth component chamber separate from the third component chamber, the fourth component chamber comprising a fourth output port defined on a distal end thereof; and
a mixing tube fluidly coupled to the third output port of the third component chamber;
a needle assembly comprising an elongate hollow stylet having a plurality of lumens therein and a needle hub having an interior cannula fluidly coupled to the elongate hollow stylet, the needle assembly removably coupled to the distal end of the first applicator in lieu of the second applicator; and
a micro seal disposed within the applicator hub of the first applicator, the micro seal configured to receive at least a portion of the mixing tube of the second applicator when the second applicator is coupled to the first applicator to establish a first fluid pathway between the first component chamber of the first applicator and the third component chamber of the second applicator separate from a second fluid pathway between the second component chamber of the first applicator and the fourth component chamber of the second applicator, and wherein the micro seal is configured to receive the interior cannula of the needle hub when the needle assembly is coupled to the first applicator to establish a fluid coupling between the first component chamber and the second component chamber with a respective one of the lumens within the elongate hollow stylet.

18. The system of claim 17, wherein the first applicator comprises a ball valve positioned adjacent the distal end of the first component chamber.

19. The system of claim 17, wherein the first applicator comprises a first male luer lock connector defined on the distal end of the first applicator, wherein the second applicator comprises a second male luer lock connector defined on the distal end of the second applicator, wherein a connector couples the distal end of the first applicator to the distal end of the second applicator, and wherein the connector comprises one or more tabs defined on a first end thereof for engaging the first male luer lock connection and one or more tabs defined on a second end thereof for engaging the second male luer lock connection.

20. The system of claim 19, wherein the connector further comprises an internal mixing chamber defined therethrough, wherein the internal mixing chamber receives contents of the first component chamber therethrough, and wherein contents of the second component chamber passes around the internal mixing chamber.
